(19) United States
(12) Patent Application Publication
(10) Pub. No.: US 2022/0242872 A1
(43) Pub. Date: Aug. 4, 2022

(54) E3 LIGASE BINDERS AND USES THEREOF

(71) Applicant: DANA-FARBER CANCER INSTITUTE, INC., Boston, MA (US)

(72) Inventors: Jun Qi, Sharon, MA (US); Eric S. Fischer, Chestnut Hill, MA (US); Paul M. Park, Waltham, MA (US); Chengkui Pei, Brighton, MA (US); Radoslaw Piotr Nowak, Boston, MA (US)

(73) Assignee: DANA-FARBER CANCER INSTITUTE, INC., Boston, MA (US)

(21) Appl. No.: 17/620,394

(22) PCT Filed: Jun. 23, 2020
(86) PCT No.: PCT/US20/39145

§ 371 Date: Dec. 17, 2021

Related U.S. Application Data
(60) Provisional application No. 62/865,626, filed on Jun. 24, 2019.

(51) Int. Cl. C07D 487/04; C07D 401/04; A61P 35/00; A61P 35/02

(52) U.S. Cl. CPC C07D 487/04; A61P 35/02; A61P 35/00; C07D 401/04

(57) ABSTRACT

Provided herein are compounds of Formulae (I) and (II), and pharmaceutically acceptable salts, solvates, hydrates, polymorphs, co-crystals, tautomers, stereoisomers, isotopically labeled derivatives, prodrugs, and compositions thereof. The compounds described herein bind an E3 ubiquitin ligase (e.g., Cereblon) and induce degradation of a transcription factor (e.g., IKZF1, IKZF3). Also provided are pharmaceutical compositions comprising the compounds, and methods of treating and/or preventing diseases (e.g., proliferative diseases (e.g., cancers (e.g., carcinoma); leukemia, lung cancer, breast cancer, liver cancer, pancreatic cancer, gastric cancer, ovarian cancer, colon cancer, colorectal cancer, multiple myeloma, and acute myeloid leukemia (AML))). Further provided are methods of inducing the degradation of a transcription factor (e.g., IKZF1, IKZF3) by administering a compound or composition described herein to a subject and/or to a biological sample (e.g., cell or tissue).

### RELATED APPLICATIONS

This application claims the benefit of priority under 35 U.S.C. § 119(e) to U.S. Provisional Application No: 62/865,626, filed on Jun. 24, 2019, which is incorporated herein by reference in its entirety.

### GOVERNMENT LICENSE RIGHTS

This invention was made with government support under grant number R01 CA214608 awarded by the National Institutes of Health. The government has certain rights in the invention.

### SEQUENCE LISTING

The instant application contains a Sequence Listing which has been submitted electronically in ASCHII format and is hereby incorporated by reference in its entirety. Said ASCII copy, created on Jun. 21, 2020, is named 52095-696001WO-SEQLISTING_ST25.txt and is 18.3 KB bytes in size.

### BACKGROUND OF THE INVENTION

Recently, a new therapeutic strategy to reduce and/or eliminate proteins associated with certain pathological states, PROTAC (proteolysis targeting chimeras; e.g., see, U.S. Patent Application Publications 2016/0058872 A1 and 2016/0176916 A1, each of which is incorporated herein by reference), was developed by creating bifunctional compounds that recruit E3 ubiquitin ligase to a target protein, which subsequently induce ubiquitination and proteasome-mediated degradation of the target protein. E3 ubiquitin ligases are proteins that, in combination with an E2 ubiquitin-conjugating enzyme, promote the attachment of ubiquitin to a lysine of a target protein via an isopeptide bond (e.g., an amide bond that is not present on the main chain of a protein). The ubiquitination of the protein results in degradation of the target protein by the proteasome.

In light of the newly identified PROTAC strategy, there is a need to identify compounds that effectively promote the degradation of target proteins, e.g., proteins found to be associated with certain pathological states, including proliferative diseases, e.g., cancers. In particular, compounds that can take advantage of cellular machinery involved in protein homeostasis (e.g., ubiquitination and proteasome degradation) to target the degradation of certain proteins may find use as therapeutic agents. Specifically there is a need for compounds that both target a target protein (e.g., transcription factors, such as IKZF1 and/or IKZF3), and also bind E3 ubiquitin ligases, resulting in ubiquitination and subsequent degradation of the target protein.

### SUMMARY OF THE INVENTION

Described herein are compounds of Formulae (I) and (II), and salts, solvates, hydrates, polymorphs, co-crystals, tautomers, stereoisomers, isotopically labeled derivatives, and prodrugs thereof, and compositions thereof. The compounds of Formulae (I) and (II), and pharmaceutically acceptable salts, solvates, hydrates, polymorphs, co-crystals, tautomers, stereoisomers, isotopically labeled derivatives, prodrugs, and compositions thereof, may induce the degradation of a target transcription factor (e.g., IKZF1 or IKZF3) in a biological sample or subject. In certain embodiments, the target transcription factor is IKZF1. In certain embodiments, the target transcription factor is IKZF3. In certain embodiments, the compounds of Formulae (I) and (II) are selective for IKZF1 compared to other transcription factors. In certain embodiments, the compounds of Formulae (I) and (II) are selective for IKZF3 compared to other transcription factors.

Also described herein are methods of using the inventive compounds, and pharmaceutically acceptable salts, solvates, hydrates, polymorphs, co-crystals, tautomers, stereoisomers, isotopically labeled derivatives, prodrugs, and compositions thereof. For example, the inventive compounds, and pharmaceutically acceptable salts, solvates, hydrates, polymorphs, co-crystals, tautomers, stereoisomers, isotopically labeled derivatives, prodrugs, and compositions thereof may be used to study the degradation of a target transcription factor (e.g., IKZF1 or IKZF3), or as therapeutics for the prevention and/or treatment of diseases associated with the target transcription factor (e.g., IKZF1 or IKZF3). The compounds described herein may be useful in treating and/or preventing a disease or condition, e.g., in treating and/or preventing a disease (e.g., proliferative disease (e.g., cancers)), in a subject in need thereof. Also provided are pharmaceutical compositions and kits including a compound described herein.

In one aspect, the present disclosure provides compounds of Formula (I):

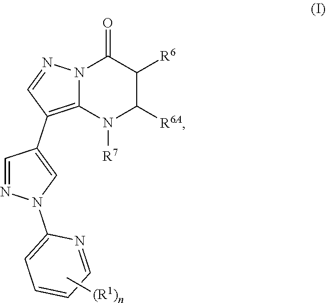

and pharmaceutically acceptable salts, solvates, hydrates, polymorphs, co-crystals, tautomers, stereoisomers, isotopically labeled derivatives, and prodrugs thereof, wherein R¹, R⁶, R^{6A}, R⁷, and n are as defined herein.

Exemplary compounds of Formula (I) include, but are not limited to:

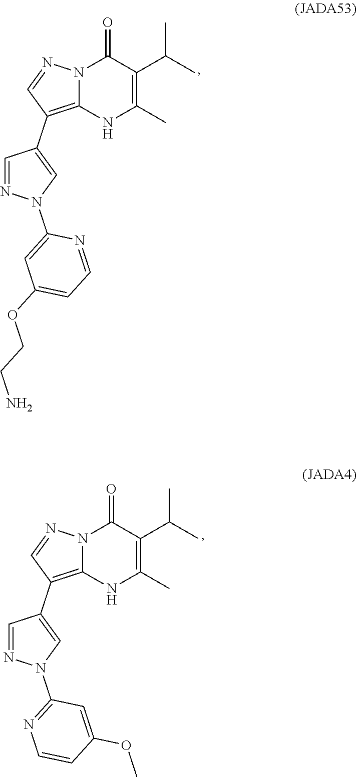 [SMILES: Cc1[nH]c2c(-c3cnn(-c4cc(OCCN)ccn4)c3)cnn2c(=O)c1C(C)C ; COc1ccnc(-n2cc(-c3cnn4c(=O)c(C(C)C)c(C)[nH]c34)cn2)c1]

In one aspect, the present disclosure provides compounds of Formula (II):

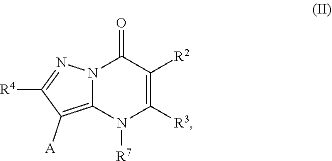

and pharmaceutically acceptable salts, solvates, hydrates, polymorphs, co-crystals, tautomers, stereoisomers, isotopically labeled derivatives, and prodrugs thereof, wherein A, R², R³, R⁴, and R⁷ are as defined herein.

Exemplary compounds of Formula (II) include, but are not limited to:

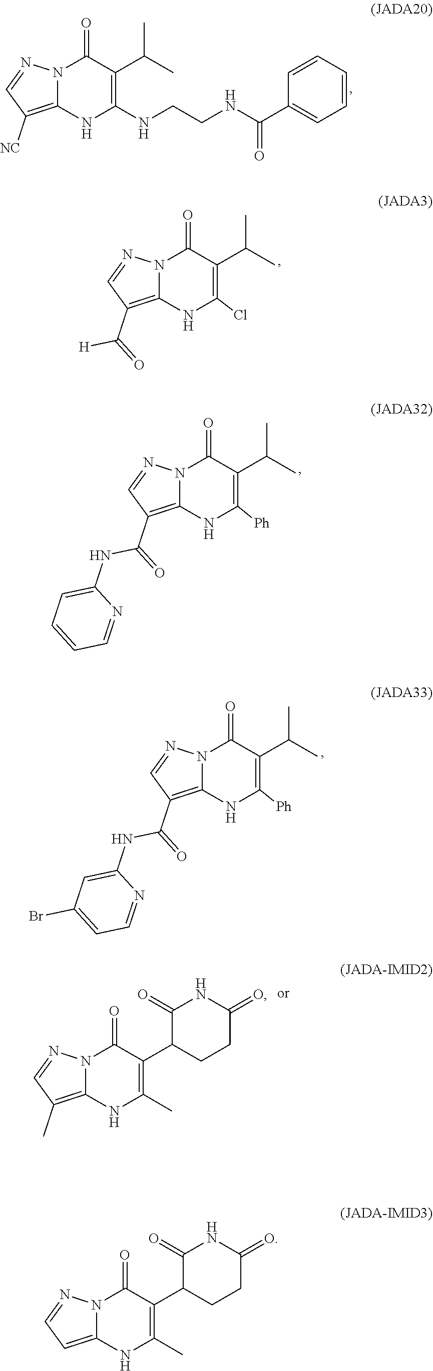

and pharmaceutically acceptable salts, solvates, hydrates, polymorphs, co-crystals, tautomers, stereoisomers, isotopically labeled derivatives, and prodrugs thereof.

Other exemplary compounds include, but are not limited to:

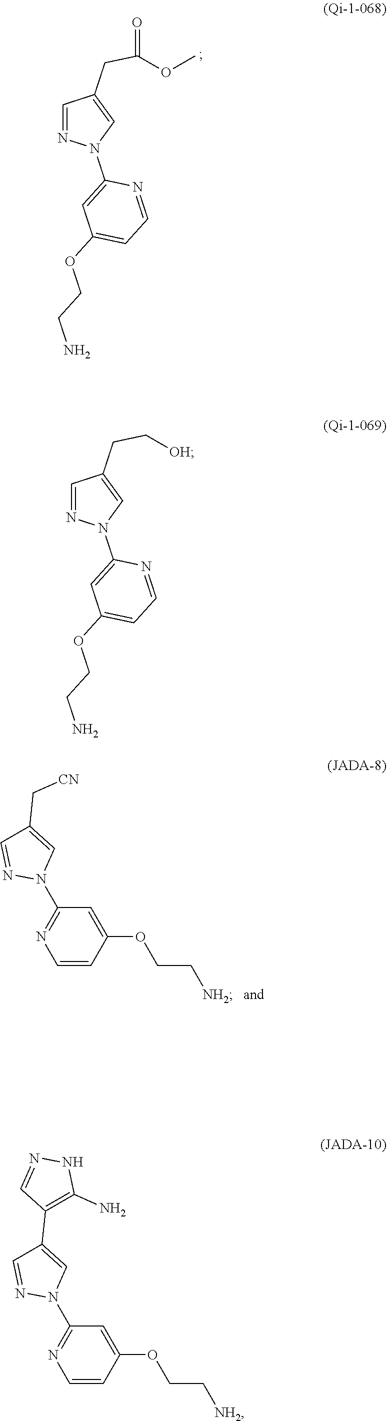 [SMILES: COC(=O)Cc1cnn(-c2cc(OCCN)ccn2)c1 ; NCCOc1ccnc(-n2cc(CCO)cn2)c1 ; N#CCc1cnn(-c2cc(OCCN)ccn2)c1 ; "and" ; NCCOc1ccnc(-n2cc(-c3cn[nH]c3N)cn2)c1]

and pharmaceutically acceptable salts, solvates, hydrates, polymorphs, co-crystals, tautomers, stereoisomers, isotopically labeled derivatives, and prodrugs thereof.

In another aspect, described herein are pharmaceutical compositions including a compound described herein, and optionally a pharmaceutically acceptable excipient. In certain embodiments, a pharmaceutical composition described herein includes a therapeutically or prophylactically effective amount of a compound described herein. The pharmaceutical compositions may be useful in inducing the degradation of a target protein, such as a transcription factor (e.g., IKZF1 or IKZF3), in a subject or biological sample (e.g., tissue, cell), in treating a disease (e.g., a proliferative disease) in a subject in need thereof, or in preventing a disease in a subject in need thereof. In certain embodiments, the compound being administered or used induces the degradation of a target transcription factor (e.g., IKZF1 or IKZF3) in a subject or biological sample (e.g., tissue or cell), in treating a disease (e.g., a proliferative disease) in a subject in need thereof, or in preventing a disease in a subject in need thereof.

In another aspect, described herein are methods for treating and/or preventing a disease (e.g., a proliferative disease). Exemplary proliferative diseases which may be treated include diseases associated with a transcription factor (e.g., IKZF1 or IKZF3). In certain embodiments, the cancer is a carcinoma. In certain embodiments, the cancer is leukemia, lung cancer (e.g., non-small cell lung cancer), breast cancer, liver cancer, pancreatic cancer, gastric cancer, ovarian cancer, colon cancer, or colorectal cancer. In other embodiments, the cancer is multiple myeloma, or acute myeloid leukemia (AML). In some embodiments, the methods entail preventing a disease in a subject in need thereof, the methods comprise administering to the subject a prophylactically effective amount of a compound or pharmaceutical composition described herein.

Another aspect relates to methods of inducing the degradation of a target transcription factor (e.g., IKZF1 or IKZF3) using a compound described herein in a biological sample (e.g., cell, tissue). In another aspect, described herein are methods of inducing the degradation of a target transcription factor (e.g., IKZF1 or IKZF3) using a compound described herein in a subject. In certain embodiments, the method involves inducing the degradation of IKZF1. In certain embodiments, the method involves inducing the degradation of IKZF3.

Described herein are methods for administering to a subject in need thereof an effective amount of a compound, or pharmaceutical composition thereof, as described herein. Also described are methods for contacting a cell with an effective amount of a compound, or pharmaceutical composition thereof, as described herein. In certain embodiments, a method described herein further includes administering to the subject an additional pharmaceutical agent. In certain embodiments, a method described herein further includes contacting the cell with an additional pharmaceutical agent (e.g., an anti-proliferative agent). In certain embodiments, the additional pharmaceutical agent is a kinase inhibitor. In certain embodiments, the additional pharmaceutical agent is an epigenetic target inhibitor (e.g., bromodomain inhibitor, DNA methylation inhibitor, histone acetyl transferase inhibitor, histone deacetylase inhibitor, protein methyltransferase inhibitor, and histone methylation inhibitor).

Another aspect of the present disclosure relates to kits comprising a container with a compound, or pharmaceutical composition thereof, as described herein. The kits described herein may include a single dose or multiple doses of the compound or pharmaceutical composition. The kits may be useful in a method or use of the disclosure. In certain embodiments, the kit further includes printed instructions for using the compound or pharmaceutical composition. A kit described herein may also include information (e.g. prescribing information) as required by a regulatory agency, such as the U.S. Food and Drug Administration (FDA).

Without intending to be bound by any theory of operation, Applicant believes that the compounds of the present invention exert their therapeutic (e.g., anti-cancer) effects or benefits by a combination of anti-proliferative and immunomodulatory effects. Mechanistically, the inventive compounds are believed to induce protein degradation, by way of forming a molecular glue with CRBN. Generally, the term “molecular glue” refers to small molecules that promote protein-protein interactions, which occur through the direct binding interactions between both protein targets with the small molecule at the protein-protein interface, or through the allosteric modification of protein structure that promotes formation of the new multiprotein complex. In the context of the present invention, the inventive compounds that act as a molecular glue in the sense that they recruit an ubiquitin ligase, which in this case is CRBN, to the target protein to function as a catalyst for targeted protein degradation. In so doing, the inventive compounds are believed to alter the substrate binding site of CRBN such that the target protein becomes a neosubstrate (Burslem et al., Chem. Rev. 117:11269-11301 (2017)). Dissociation of the molecular glue after the ubiquitination step enables subsequent function on a different molecule of the target protein (Che et al., Bioorg. Med. Chem. Lett. 28:2585-2592 (2018)).

The details of embodiments of the invention are set forth herein. Other features, objects, and advantages of the invention will be apparent from the Detailed Description, Examples, Figures, and Claims.

### DEFINITIONS

Definitions of specific functional groups and chemical terms are described in more detail below. The chemical elements are identified in accordance with the Periodic Table of the Elements, CAS® version, Handbook of Chemistry and Physics, 75^{th} E a inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGrawHill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain. For example, hydrocarbon chain —C^{A}H(C^{B}H₂C^{C}H₃)— includes only one carbon unit C^{A}. The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

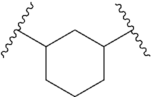 [SMILES: *C1CCCC(*)C1]

is a C₃ hydrocarbon chain. When a range of values is used, e.g., a C₁₋₆ hydrocarbon chain, the meaning of the range is as described herein. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C═C—CH₂—, and —C≡C—CH═CH— are all examples of a unsubstituted and unsaturated hydrocarbon chain. In certain embodiments, the hydrocarbon chain is unsubstituted (e.g., —(CH₂)₄)—. In certain embodiments, the hydrocarbon chain is substituted (e.g., —CH(C₂H₅)— and —CF₂)—. Any two substituents on the hydrocarbon chain may be joined to form an optionally substituted carbocyclyl, optionally substituted heterocyclyl, optionally substituted aryl, or optionally substituted heteroaryl ring. For instance,

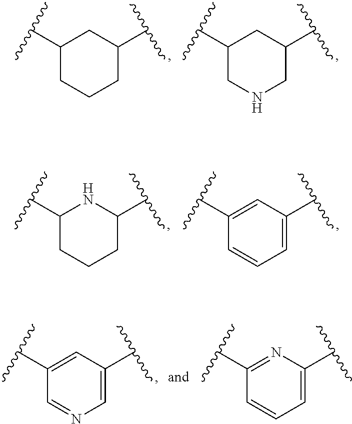 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments

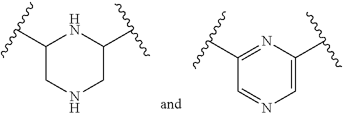 [SMILES: *C1CNCC(*)N1 ; *c1cncc(*)n1]

are not within the scope of the hydrocarbon chains described herein.

“Alkyl” refers to a radical of a straightchain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3methyl-2butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

“Alkenyl” refers to a radical of a straightchain or branched hydrocarbon group having from 2 to 20 carbon atoms, one or more carboncarbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon carbon double bonds can be internal (such as in 2butenyl) or terminal (such as in 1-butenyl). Examples of C₂₋₄ alkenyl groups include ethenyl (C₂), 1propenyl (C₃), 2propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkenyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and -zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3to 10membered non aromatic ring system having ring carbon atoms and 1 to 4 ring heteroatoms, wherein each heteroatom is independently selected from the group consisting of nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered nonaromatic ring system having ring carbon atoms and 1-4 ring heteroatoms, wherein each heteroatom is independently selected from the group consisting of nitrogen, oxygen, sulfur, boron, phosphorus, and silicon (“5-10 membered heterocyclyl”). In some embodiments, a heterocyclyl group is a 5-8 membered nonaromatic ring system having ring carbon atoms and 1-4 ring heteroatoms, wherein each heteroatom is independently selected from the group consisting of nitrogen, oxygen, and sulfur (“5-8 membered heterocyclyl”). In some embodiments, a heterocyclyl group is a 5-6 membered nonaromatic ring system having ring carbon atoms and 1-4 ring heteroatoms, wherein each heteroatom is independently selected from the group consisting of nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahy drofuranyl, dihy drofuranyl, tetrahy drothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from the group consisting of nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from the group consisting of nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from the group consisting of nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from the group consisting of nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

The term “optionally substituted” refers to substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa}, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂—NR^{bb}P(═O)(N(R^{bb})₂)₂—P(R^{cc})₂—P(OR^{cc})₂—P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(+O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)2⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄9 ⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)₂(R^{X1})₂, —C(═S)R^{X1}, —C(═S)N(R^{X1})₂, and —C(═S)S(R^{X1}), —C(═NR^{X1})OR^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: —O—alkyl.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₁₀ alkenyl, C₁₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)] methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylypethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2cyanoethyl carbamate, m-chloropacyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(onitrophenyl)methyl carbamate, tamyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2one, 5-substituted 1,3-dibenzyl-1,3,5-triazacyclohexan-2one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di-(4methoxyphenyl)methylamine, N-5-dibenzosuberylamine, N-triphenylmethylamine (Tr), N[-(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N′,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, onitrobenzenesulfenamide (Nps), 2,4dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═C)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb}) ₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4′-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tripxylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio)ethyl carbonate (Peoc), alkyl isobutyl carbonate, alkyl vinyl carbonate alkyl allyl carbonate, alkyl p-nitrophenyl carbonate, alkyl benzyl carbonate, alkylpmethoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis-(1,1-dimethylpropyl)phenoxyacetate, chlorodiphenylacetate, isobutyrate, monosuccinoate, (E)-2-methyl-2-butenoate, o-(methoxyacyl)benzoate, anaphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S (═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in a heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6^{th} ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, amines, ammonia, alcohols, ether moieties, sulfur-containing moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “isotopically enriched derivative” refers to a derivative of the compound that contains a distribution of mass isotopes different from the naturally occurring isotopic distribution, whereby one of the mass isotopes has an enrichment level higher than present in the naturally occurring level.

The term “prodrugs” refer to compounds, including derivatives of the compounds of Formula (I) or (II), which have cleavable groups and become by solvolysis or under physiological conditions the compounds of Formula (I) or (II) which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases, it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formulae (I) and (II) may be preferred.

A “subject” to which administration is contemplated includes, but is not limited to, humans (i.e., a male or female of any age group, e.g., a pediatric subject (e.g., infant, child, adolescent) or adult subject (e.g., young adult, middleaged adult, or senior adult)) and/or other nonhuman animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A nonhuman animal may be a transgenic animal.

The terms “administer,” “administering,” or “administration,” refers to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for binding a target (e.g., a transcription factor (e.g., IKZF1 or IKZF3)) (and/or inducing the degradation of the target (e.g., a transcription factor (e.g., IKZF1 or IKZF3)), and/or inducing downstream effects (e.g., effects in transcription) from binding a target.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a target (e.g., a transcription factor (e.g., IKZF1 or IKZF3)). In certain embodiments, a prophylactically effective amount is an amount sufficient for treating a proliferative disease (e.g., cancer). In certain embodiments, a prophylactically effective amount is an amount sufficient for binding a target (e.g., a transcription factor (e.g., IKZF1 or IKZF3)) and/or inducing the degradation of the target (e.g., a transcription factor (e.g., IKZF1 or IKZF3)) and preventing recurrence of a proliferative disease (e.g., cancer).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used interchangeably and refer to an abnormal mass of tissue wherein the growth of the mass surpasses and is not coordinated with the growth of normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e. g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity, such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The term “E3 ubiquitin ligase” or “E3 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate.

An exemplary E3 ubiquitin ligase amino acid sequence from GenBank, ACH72645.1 (Homo sapiens), incorporated herein by reference, is set forth below:

(SEQ ID NO: 1)
MESGGRPSLC QFILLGTTSV VTAALYSVYR QKARVSQELK
GAKKVHLGED LKSILSEAPG KCVPYAVIEG AVRSVKETLN
SQFVENCKGV IQRLTLQEHK MVWNRTTHLW NDCSKIIHQR
TNTVPFDLVP HEDGVDVAVR VLKPLDSVDL GLETVYEKFH
PSIQSFTDVI GHYISGERPK GIQETEEMLK VGATLTGVGE
LVLDNNSVRL QPPKQGMQYY LSSQDFDSLL QRQESSVRLW
KVLALVFGFA TCATLFFILR KQYLQRQERL RLKQMQEEFQ
EHEAQLLSRA KPEDRESLKS ACVVCLSSFK SCVFLECGHV
CSCTECYRAL PEPKKCPICR QAITRVIPPY NS.

Another exemplary E3 ubiquitin ligase amino acid sequence from GenBank, AAP47175.1 (Homo sapiens), incorporated herein by reference, is set forth below:

(SEQ ID NO: 2)
MEEGNNNEEV IHLNNFHCHR GQEWINLRDG PITISDSSDE
ERIPMLVTPA PQQHEEEDLD DDVILTETNK PQRSRPNLIK
PAAQWQDLKR LGEERPKKSR AAFESDKSSY FSVCNNPLFD
SGAQDDSEDD YGEFLDLGPP GISEFTKPSG QTEREPKPGP
SHNQAANDIV NPRSEQKVII LEEGSLLYTE SDPLETQNQS
SEDSETELLS NLGESAALAD DQAIEEDCWL DHPYFQSLNQ
QPREITNQVV PQERQPEAEL GRLLFQHEFP GPAFPRPEPQ
QGGISGPSSP QPAHPLGEFE DQQLASDDEE PGPAFPMQES
QEPNLENIWG QEAAEVDQEL VELLVKETEA RFPDVANGFI
EEIIHFKNYY DLNVLCNFLL ENPDYPKRED RIIINPSSSL
LASQDETKLP KIDFFDYSKL TPLDQRCFIQ AADLLMADFK
VLSSQDIKWA LHELKGHYAI TRKALSDAIK KWQELSPETS
GKRKKRKQMN QYSYIDFKFE QGDIKIEKRM FFLENKRRHC
RSYDRRALLP AVQQEQEFYE QKIKEMAEHE DFLLALQMNE
EQYQKDGQLI ECRCCYGEFP FEELTQCADA HLFCKECLIR
YAQEAVFGSG KLELSCMEGS CTCSFPTSEL EKVLPQTILY
KYYERKAEEE VAAAYADELV RCPSCSFPAL LDSDVKRFSC
PNPHCRKETC RKCQGLWKEH NGLTCEELAE KDDIKYRTSI
EEKMTAARIR KCHKCGTGLI KSEGCNRMSC RCGAQMCYLC
RVSINGYDHF CQHPRSPGAP CQECSRCSLW TDPTEDDEKL
IEEIQKEAEE EQKRKNGENT FKRIGPPLEK PVEKVQRVEA
LPRPVPQNLP QPQMPPYAFA HPPFPLPPVR PVFNNFPLNM
GPIPAPYVPP LPNVRVNYDF GPIHMPLEHN LPMHFGPQPR HRF.

Another exemplary E3 ubiquitin ligase amino acid sequence from GenBank, AAP47174.1 (Homo sapiens), incorporated herein by reference, is set forth below:

(SEQ ID NO: 3)
MEEGNNNEEV IHLNNFHCHR GQEWINLRDG PITISDSSDE
ERIPMLVTPA PQQHEEEDLD DDVILTEDDS EDDYGEFLDL
GPPGISEFTK PSGQTEREPK PGPSHNQAAN DIVNPRSEQK
VIILEEGSLL YTESDPLETQ NQSSEDSETE LLSNLGESAA
LADDQAIEED CWLDHPYFQS LNQQPREITN QVVPQERQPE
AELGRLLFQH EFPGPAFPRP EPQQGGISGP SSPQPAHPLG
EFEDQQLASD DEEPGPAFPM QESQEPNLEN IWGQEAAEVD
QELVELLVKE TEARFPDVAN GFIEEIIHFK NYYDLNVLCN
FLLENPDYPK REDRIIINPS SSLLASQDET KLPKIDFFDY
SKLTPLDQRC FIQAADLLMA DFKVLSSQDI KWALHELKGH
YAITRKALSD AIKKWQELSP ETSGKRKKRK QMNQYSYIDF
KFEQGDIKIE KRMFFLENKR RHCRSYDRRA LLPAVQQEQE
FYEQKIKEMA EHEDFLLALQ MNEEQYQKDG QLIECRCCYG
EFPFEELTQC ADAHLFCKEC LIRYAQEAVF GSGKLELSCM
EGSCTCSFPT SELEKVLPQT ILYKYYERKA EEEVAAAYAD
ELVRCPSCSF PALLDSDVKR FSCPNPHCRK ETCRKCQGLW
KEHNGLTCEE LAEKDDIKYR TSIEEKMTAA RIRKCHKCGT
GLIKSEGCNR MSCRCGAQMC YLCRVSINGY DHFCQHPRSP
GAPCQECSRC SLWTDPTEDD EKLIEEIQKE AEEEQKRKNG
ENTFKRIGPP LEKPVEKVQR VEALPRPVPQ NLPQPQMPPY
AFAHPPFPLP PVRPVFNNFP LNMGPIPAPY VPPLPNVRVN
YDFGPIHMPL EHNLPMHFGP QPRHRF.

Human “Cereblon” (CRBN) is a protein of 442 amino acids with an apparent molecular weight of ˜51 kDa (GenBank: AAH17419). (For the CRBN protein sequence, see: Higgins et al., Neurology. 2004, 63, 1927-31. For additional information related to the CRBN structure, see, Hartmann et al., PLoS One. 2015, 10, e0128342). Human CRBN contains the N-terminal part (237-amino acids from 81 to 317) of ATP-dependent Lon protease domain without the conserved Walker A and Walker B motifs, 11 casein kinase II phosphorylation sites, four protein kinase C phosphorylation sites, one N-linked glycosylation site, and two myristoylation sites. CRBN is widely expressed in testis, spleen, prostate, liver, pancreas, placenta, kidney, lung, skeletal muscle, ovary, small intestine, peripheral blood leukocyte, colon, brain, and retina. CRBN is located in the cytoplasm, nucleus, and plasma membrane (e.g., peripheral membrane) in the brain and other tissues. (Chang et al., Int. J. Biochem. Mol. Biol. 2011, 2, 287-94)

Cereblon is an E3 ubiquitin ligase, and it forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

The term “transcription factor” refers to a protein involved in the process of converting, or transcribing, DNA into RNA. Transcription factors include a diverse range of proteins that initiate and regulate the transcription of genes. Transcription factors bind to DNA and assist in controlling gene expression. Exemplary transcription factors include Ikaros family zinc finger protein 1 (“IKZF1”) and Ikaros family zinc finger protein 3 (“IKZF3”). For IKZF1 (Homo sapiens), exemplary sequences from GenBank are of Genbank ID AAH18349. For IKZF3 (Homo sapiens), exemplary sequences from GenBank are of Genbank ID AAH32707.

The term “binder” refers to a compound that binds to a protein. The binder binds to a protein with a K_{d} of less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

The term “proteasome” refers to a protease complex for carrying out degradation of proteins. Specifically, the proteasome is a multi-subunit enzyme complex, which can also play a key role regulating proteins that control cell-cycle progression and apoptosis. The proteasome conducts proteolysis of selected proteins.

### BRIEF DESCRIPTION OF THE DRAWINGS

FIG. 1 shows results of a CRBN Fluorescence Polarization (FP) displacement assay of tested exemplary compounds Qi-1-068, Qi-1-069, JADA53, and positive control lenalidomide (Len) at different concentrations [M], measured in [M] per mP, as well as the chemical structures of exemplary compounds Qi-1-068, Qi-1-069, JADA53, and positive control lenalidomide (Len).

FIG. 2 shows results of the CRBN Alpha binding assay of tested exemplary compounds Qi-1-068, Qi-1-069, JADA53, and positive control lenalidomide (Len) at different concentrations [M], as well as the chemical structures of exemplary compounds JADA53 and lenalidomide.

FIG. 3 shows results of an assay for CRBN binding (and degradation of IKZF or actin) upon treatment for 24 hours with exemplary compounds Qi-01-68, JADA53, and positive control lenalidomide (at the indicated concentrations in μM) in 293T WT cells (Homo sapiens kidney cell line; wild type) (cell: Jurkat W T; exemplary compounds for treatment: Qi-01-68, JADA53, and positive control Lenalidomide; time point: 24 hours). Exemplary compound 53 (JADA53) demonstrates good binding of target CRBN in the cell, where binding with CRBN in the cell can degrade IKZF. According to the CRBN FP displacement assay (FIG. 1), exemplary compound Qi-01-68 has the same binding activity as JADA53, however, it does not degrade IKZF1 in cell (FIG. 3).

FIG. 4 shows the results of a CRBN Fluorescence Polarization (FP) displacement assay of tested exemplary compounds JADA51, JADA52, JADA53, JADA54, KDMS c70, and positive control lenalidomide at different concentrations [M], measured in [M] per mP, as well as the chemical structures of exemplary compounds JADA52, JADA53, and positive control lenalidomide.

FIG. 5 shows the results of 24 hour IKZF Western Blots of assays of the treatment with exemplary compounds JADA3, JADA32, JADA53, and positive control lenalidomide at different indicated concentrations [μM], and the degradation of IKZF1, IKZF2, IKZF3, and actin in Jurkat cells, for a 24 hour period.

FIG. 6A shows the results of a CRBN Fluorescence Polarization (FP) displacement assay of tested exemplary compounds JADA3, JADA4, JADA20, JADA32, JADA33, JADA35, JADA36, JADA52, JADA53, JADA62, JADA63, JADA65, and positive control lenalidomide at different concentrations [M], measured in [M] per Mp. FIG. 6B shows the chemical structures of exemplary compounds JADA3, JADA4, JADA20, JADA32, and JADA33.

FIG. 7 shows the results of a CRBN Fluorescence Polarization (FP) displacement assay of tested exemplary compounds PP-29, PP-30, PP-59, PP-60, PP-64, PP-65, JADA53, and positive control lenalidomide at different concentrations [M], measured in [M] per Mp.

FIG. 8 shows the results of a 24-hour IKZF-GFP (green fluorescent protein) degradation assay upon treatment with tested exemplary compounds PP-29, PP-30, PP-59, PP-60, PP-64, PP-65, JADA53, positive control Lenalidomide, and control without DMSO at different concentrations [M].

FIG. 9 shows the chemical structures of exemplary compounds JADA53 (IKZF1 degrader) and exemplary IZKF1 degraders JADA-IMID2 and JADA-IMID3.

FIG. 10 shows the results of TMT LC-MS3 mass spectrometry experiments examining fold change in relative abundance comparing treatment with exemplary compound JADA53 to DMSO control treatment in Kelly cells (Kelly cells - JADA53; 1 μM concentration, 6 hours, ×1).

FIG. 11 shows the results of TMT LC-MS3 mass spectrometry experiments examining fold change in relative abundance comparing treatment with exemplary compound JADA53 to DMSO control treatment in MM.1 human multiple myeloma (MM) cell lines (MM1s-JADA53; 10 μM concentration, 12 hours, ×1).

FIG. 12 shows the results of TMT LC-MS3 mass spectrometry experiments examining fold change in relative abundance comparing treatment with exemplary compound JADA53 to DMSO control treatment in MM.1 human multiple myeloma (MM) cell lines (MM1s-JADA53; 10 μM concentration, 24 hours, ×1).

FIG. 13 shows results of the CRBN Alpha binding assay of tested exemplary compounds Qi-1-068 (Qi68), Qi-1-069 (Qi69), JADA53, and JADA54, JADA53 intermediates JADA-18 and JADA-10, and positive control lenalidomide (Len) at different concentrations [M], as well as the chemical structures of the JADA53 intermediates JADA-18 and JADA-10.

### DETAILED DESCRIPTION OF CERTAIN EMBODIMENTS OF THE INVENTION

The compounds described herein interact with an E3 ubiquitin ligase and a target transcription factor (e.g., IKZF1 or IKZF3). As described herein, without wishing to be bound by any particular theory, the therapeutic effect may be the result of degradation, modulation, or binding of an E3 ubiquitin ligase (e.g., Cereblon) by a compound described herein. For example, the therapeutic effect may be a result of recruitment of an E3 ubiquitin ligase (e.g., Cereblon) which induces the ubiquitination of a target transcription factor (e.g., IKZF1 or IKZF3). This leads to the ubiquitination of the target transcription factor and its subsequent degradation by the proteasome. The compounds can also be used in place of current E3 ubiquitin ligase binders (e.g., thalidomide, lenalidomide) and attached via a linker to a target protein binding moiety, which may be useful in promoting the degradation of the target protein.

A compound may be provided for use in any composition, kit, or method described herein as a pharmaceutically acceptable salt, co-crystal, tautomer, stereoisomer, solvate, hydrate, polymorph, isotopically enriched derivative, or prodrug thereof

In one aspect, disclosed are compounds of Formula (I):

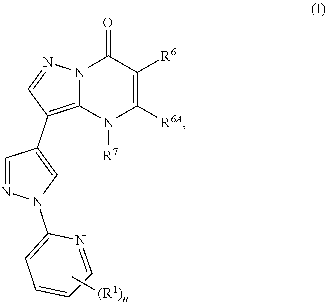

or a pharmaceutically acceptable salt, co-crystal, tautomer, stereoisomer, solvate, hydrate, polymorph, isotopically enriched derivative, or prodrug thereof, wherein:

each instance of R¹ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, or —O(CH₂)ₓR^{1A};

provided at least one instance of R¹ is —O(CH₂)ₓR^{1A};

R⁶ is unsubstituted isopropyl, —(CH₂)C(═O)OMe, or —(CH₂)₂OH;

R^{6A} is optionally substituted alkyl;

R⁷ is hydrogen, optionally substituted acyl, optionally substituted alkyl, or a nitrogen protecting group; R^{1A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —N(R^{B})₂, or —SR^{A}, as valency permits;

each instance of R^{A} is independently hydrogen, optionally substituted acyl, optionally substituted alkyl, an oxygen protecting group when attached to an oxygen atom, or a sulfur protecting group when attached to a sulfur atom;

each instance of R^{B} is independently hydrogen, optionally substituted acyl, optionally substituted alkyl, or a nitrogen protecting group;

n is 1, 2, 3, or 4; and

x is 0, 1, 2, 3, 4, 5, or 6, provided that when R⁶ is isopropyl, R^{6A} is methyl, R⁷ is hydrogen, and n is 1, R¹ is not methyl or hydroxymethyl.

In certain embodiments, the compound of Formula (I) is represented by any one of formulae:

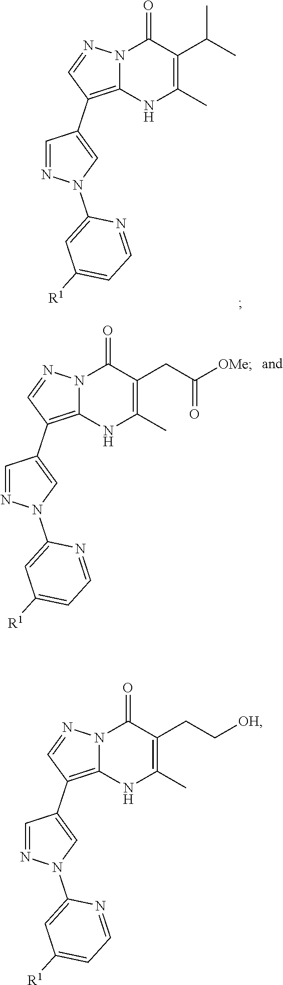

or a pharmaceutically acceptable salt, co-crystal, tautomer, stereoisomer, solvate, hydrate, polymorph, isotopically enriched derivative, or prodrug thereof.

In certain embodiments, n is 1. In certain embodiments, n is 2. In certain embodiments, n is 3. In certain embodiments, n is 4. In Formula (I), at least one instance of R¹ is —O(CH₂)ₓR^{1A}, wherein each instance of R^{1A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —N(R^{B})₂, or —SR^{A}, as valency permits; and x is 0, 1, 2, 3, 4, 5, or 6; and R^{A} and R^{B} are as defined herein. In certain embodiments, x is 0. In certain embodiments, x is 1. In certain embodiments, x is 2. In certain embodiments, x is 3. In certain embodiments, x is 4. In certain embodiments, x is 5. In certain embodiments, x is 6. In certain embodiments, at least one instance of R¹ is —O(CH₂)₂N(RB)₂. In certain embodiments, at least one instance of R¹ is —O(CH₂)₂NH₂. In certain embodiments, at least one instance of R¹ is —OMe. In certain embodiments, R^{1A} is hydrogen. In certain embodiments, R^{1A} is optionally substituted acyl (e.g., —C(═O)Me). In certain embodiments, R^{1A} is optionally substituted alkyl (e.g., substituted or unsubstituted C₁₋₆ alkyl). In certain embodiments, R^{1A} is substituted or unsubstituted methyl. In certain embodiments, R^{1A} is substituted or unsubstituted ethyl. In certain embodiments, R^{1A} is substituted or unsubstituted propyl. In certain embodiments, R^{1A} is alkenyl (e.g., substituted or unsubstituted C₂₋₆ alkenyl). In certain embodiments, R^{1A} is optionally substituted alkynyl (e.g., substituted or unsubstituted C₂₋₆ alkynyl). In certain embodiments, R^{1A} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{1A} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{1A} is optionally substituted aryl (e.g., substituted or unsubstituted, 6- to 10-membered aryl). In certain embodiments, R^{1A} is optionally substituted benzyl. In certain embodiments, R^{1A} is optionally substituted phenyl. In certain embodiments, R^{1A} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{1A} is —OR^{A} (e.g., —OMe). In certain embodiments, R^{1A} is —N(R^{B})₂ (e.g., —NH₂). In certain embodiments, R^{1A} is —SR^{A} (e.g., —SMe). In certain embodiments, R^{1A} is —OR^{A}, —N(R^{B})₂, or —SR^{A}, as valency permits, wherein R^{A} and R^{B} are as defined herein. In certain embodiments, at least one instance of R¹ is —OMe.

In certain embodiments, at least one instance of R¹ is halogen (e.g., F, Cl, Br, or I). In certain embodiments, at least one instance of R¹ is F. In certain embodiments, at least one instance of R¹ is Cl. In certain embodiments, at least one instance of R¹ is Br. In certain embodiments, at least one instance of R¹ is I. In certain embodiments, at least one instance of R¹ is optionally substituted acyl (e.g., —C(═O)Me). In certain embodiments, at least one instance of R¹ is optionally substituted alkyl (e.g., substituted or unsubstituted C₁₋₆ alkyl). In certain embodiments, at least one instance of R¹ is substituted or unsubstituted methyl. In certain embodiments, at least one instance of R¹ is substituted or unsubstituted ethyl. In certain embodiments, at least one instance of R¹ is substituted or unsubstituted n-propyl. In certain embodiments, at least one instance of R¹ is unsubstituted isopropyl. In certain embodiments, at least one instance of R¹ is unsubstituted isopropyl. In certain embodiments, at least one instance of R¹ is alkenyl (e.g., substituted or unsubstituted C₂₋₆ alkenyl). In certain embodiments, at least one instance of R¹ is optionally substituted alkynyl (e.g., substituted or unsubstituted C₂₋₆ alkynyl). In certain embodiments, at least one instance of R¹ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R¹ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R¹ is optionally substituted aryl (e.g., substituted or unsubstituted, 6- to 10-membered aryl). In certain embodiments, at least one instance of R¹ is optionally substituted benzyl. In certain embodiments, at least one instance of R¹ is optionally substituted phenyl. In certain embodiments, at least one instance of R¹ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R¹ is —CN.

In certain embodiments, at least a second instance of R¹ is —O(CH₂)ₓR^{1A}, wherein each instance of R^{1A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —N(R^{B})₂, or —SR^{A}, as valency permits; and x is 0, 1, 2, 3, 4, 5, or 6; and R^{A} and R^{B} are as defined herein.

In certain embodiments, at least one instance of R^{A} is hydrogen. In certain embodiments, at least one instance of R^{A} is substituted or unsubstituted acyl (e.g., —C(═O)Me). In certain embodiments, at least one instance of R^{A} is optionally substituted alkyl (e.g., substituted or unsubstituted C₁₋₆ alkyl). In certain embodiments, at least one instance of R^{A} is substituted or unsubstituted C₁₋₆ alkyl. In certain embodiments, at least one instance of R^{A} is substituted or unsubstituted methyl. In certain embodiments, at least one instance of R^{A} is substituted or unsubstituted ethyl. In certain embodiments, at least one instance of R^{A} is substituted or unsubstituted propyl. In certain embodiments, at least one instance of R^{A} is an oxygen protecting group when attached to an oxygen atom. In certain embodiments, at least one instance of R^{A} is a sulfur protecting group when attached to a sulfur atom.

In certain embodiments, at least one instance of R^{B} is hydrogen. In certain embodiments, two instances of R^{B} are hydrogen. In certain embodiments, at least one instance of R^{B} is substituted or unsubstituted acyl (e.g., —C(═O)Me). In certain embodiments, at least one instance of R^{B} is optionally substituted alkyl (e.g., substituted or unsubstituted C₁₋₆ alkyl). In certain embodiments, at least one instance of R^{B} is unsubstituted C₁₋₆ alkyl. In certain embodiments, two instances of R^{B} are unsubstituted C₁₋₆ alkyl. In certain embodiments, two instances of R^{B} are substituted or unsubstituted C₁₋₆ alkyl. In certain embodiments, at least one instance of R^{B} is substituted or unsubstituted methyl. In certain embodiments, at least one instance of R^{B} is substituted or unsubstituted ethyl. In certain embodiments, at least one instance of R^{B} is substituted or unsubstituted propyl. In certain embodiments, at least one instance of R^{B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of RB are each a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, at least one instance of R¹ is —O(CH₂)₂NH₂. In certain embodiments, at least one instance of R¹ is —O(CH₂)₂NH(optionally substituted alkyl). In certain embodiments, at least one instance of R¹ is —O(CH₂)₂N(optionally substituted alkyl)_{2.}

In certain embodiments, R⁶ is unsubstituted isopropyl. In certain embodiments, R⁶ is —(CH₂)C(═O)OMe. In certain embodiments, R⁶ is —(CH₂)₂OH.

In certain embodiments, R^{6A} is optionally substituted alkyl (e.g., substituted or unsubstituted C₁₋₆ alkyl). In certain embodiments, R^{6A} is unsubstituted C₁₋₆ alkyl. In certain embodiments, R^{6A} is substituted or unsubstituted methyl. In certain embodiments, R^{6A} is substituted methyl. In certain embodiments, R^{6A} is unsubstituted methyl. In certain embodiments, R^{6A} is substituted or unsubstituted ethyl. In certain embodiments, R^{6A} is substituted or unsubstituted propyl.

Formulae (I) and (II) both include substituent R⁷. In certain embodiments, R⁷ is hydrogen. In certain embodiments, R⁷ is optionally substituted acyl (e.g., —C(═O)Me). In certain embodiments, R⁷ is optionally substituted alkyl (e.g., substituted or unsubstituted C₁₋₆ alkyl). In certain embodiments, R⁷ is substituted or unsubstituted methyl. In certain embodiments, R⁷ is substituted or unsubstituted ethyl. In certain embodiments, R⁷ is substituted or unsubstituted propyl. In certain embodiments, R⁷ is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, R⁶ is unsubstituted isopropyl and R^{6A} is unsubstituted methyl. In certain embodiments, R⁶ is unsubstituted isopropyl, R^{6A} is unsubstituted methyl, and R⁷ is hydrogen. In certain embodiments, R⁶ is unsubstituted isopropyl, R^{6A} is unsubstituted methyl, R⁷ is hydrogen, and n is 1.

In certain embodiments, the compound of Formula (I) is represented by any one of structures:

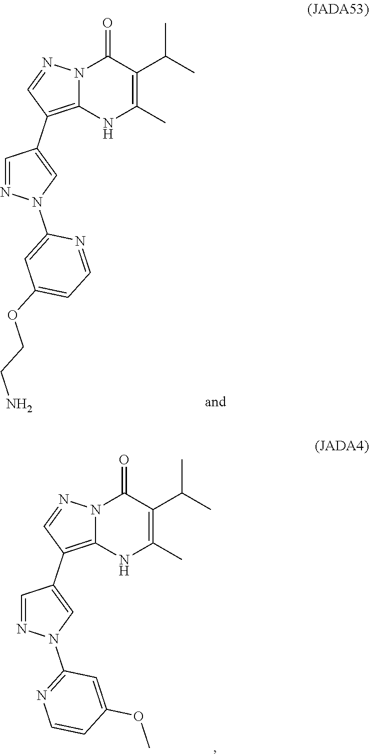 [SMILES: Cc1[nH]c2c(-c3cnn(-c4cc(OCCN)ccn4)c3)cnn2c(=O)c1C(C)C ; COc1ccnc(-n2cc(-c3cnn4c(=O)c(C(C)C)c(C)[nH]c34)cn2)c1]

or a pharmaceutically acceptable salt, solvate, hydrate, polymorph, co-crystal, tautomer, stereoisomer, isotopically labeled derivative, or prodrug thereof.

In certain embodiments, the compound of Formula (I) is not any one of the following structures:

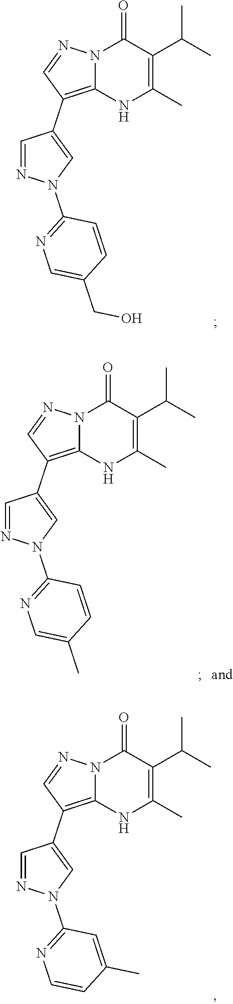 [SMILES: Cc1[nH]c2c(-c3cnn(-c4ccc(CO)cn4)c3)cnn2c(=O)c1C(C)C ; Cc1ccc(-n2cc(-c3cnn4c(=O)c(C(C)C)c(C)[nH]c34)cn2)nc1 ; Cc1ccnc(-n2cc(-c3cnn4c(=O)c(C(C)C)c(C)[nH]c34)cn2)c1]

or a pharmaceutically acceptable salt, solvate, hydrate, polymorph, co-crystal, tautomer, stereoisomer, isotopically labeled derivative, or prodrug thereof.

In another aspect, disclosed are compounds of Formula (II):

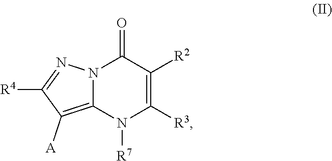

or a pharmaceutically acceptable salt, co-crystal, tautomer, stereoisomer, solvate, hydrate, polymorph, isotopically enriched derivative, or prodrug thereof, wherein:

A is hydrogen, optionally substituted alkyl, —C(═O)N(R⁵)₂, —C(═O)(R⁵), —C(═O)OR⁵, or —CN;

R² is hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{2B}, —N(R^{2A})₂, or —SR^{2B};

R³ is hydrogen, halogen, optionally substituted acyl, optionally substituted alkyl, optionally substituted alkenyl, optionally substituted alkynyl, optionally substituted carbocyclyl, optionally substituted aryl, optionally substituted heteroaryl, —OR^{2A}, —N(R^{2B})₂, or —SR^{2A};

R⁴ is hydrogen, halogen, optionally substituted acyl, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{2A}, —N(R^{2B})², —SR^{2A}, or —CN; and

each instance of R⁵ is independently hydrogen, optionally substituted alkyl, optionally substituted alkenyl, optionally substituted alkynyl, optionally substituted carbocyclyl, optionally substituted aryl, optionally substituted heterocyclyl, or optionally substituted heteroaryl;

R⁷ is hydrogen, optionally substituted acyl, optionally substituted alkyl, or a nitrogen protecting group;

each instance of R^{2A} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and

each instance of R^{2B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or optionally two instances of R^{2A} are taken together with their intervening atoms to form a substituted or unsubstituted heterocyclic or substituted or unsubstituted heteroaryl ring.

In certain embodiments, A is hydrogen. In certain embodiments, A is optionally substituted alkyl (e.g., optionally substituted C₁₋₆ alkyl). In certain embodiments, A is optionally substituted C₁₋₆ alkyl. In certain embodiments, A is substituted or unsubstituted methyl. In certain embodiments, A is unsubstituted methyl. In certain embodiments, A is substituted or unsubstituted ethyl. In certain embodiments, A is substituted or unsubstituted propyl.

In certain embodiments, A is —C(═O)N(R⁵)₂, wherein each instance of R⁵ is independently hydrogen, optionally substituted alkyl, optionally substituted alkenyl, optionally substituted alkynyl, optionally substituted carbocyclyl, optionally substituted aryl, optionally substituted heterocyclyl, or optionally substituted heteroaryl. In certain embodiments, A is hydrogen, —C(═O)H, —C(═O)NH(optionally substituted heteroaryl), optionally substituted C₁₋₆ alkyl, or —CN. In certain embodiments, A is hydrogen, —C(═O)H,

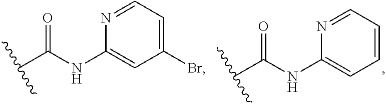

unsubstituted methyl, or —CN.

In certain embodiments, at least one instance of R⁵ is hydrogen. In certain embodiments, at least one instance of R⁵ is optionally substituted alkyl (e.g., substituted or unsubstituted C₁₋₆ alkyl). In certain embodiments, at least one instance of R⁵ is substituted or unsubstituted methyl. In certain embodiments, at least one instance of R⁵ is substituted or unsubstituted ethyl. In certain embodiments, at least one instance of R⁵ is substituted or unsubstituted propyl. In certain embodiments, at least one instance of R⁵ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R⁵ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R⁵ is optionally substituted aryl (e.g., substituted or unsubstituted, 6- to 10-membered aryl). In certain embodiments, at least one instance of R⁵ is optionally substituted benzyl. In certain embodiments, at least one instance of R⁵ is optionally substituted phenyl. In certain embodiments, at least one instance of R⁵ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R⁵ is optionally substituted 5- to 6-membered, monocyclic heteroaryl, wherein one, two, three, or four atoms in the heteroaryl ring system are independently nitrogen, oxygen, or sulfur. In certain embodiments, at least one instance of R⁵ is optionally substituted pyridinyl. In certain embodiments, at least one instance of R⁵ is optionally substituted pyridazinyl, optionally substituted pyrimidinyl, or optionally substituted pyrazinyl.

In certain embodiments, A is —C(═O)NH(optionally substituted heteroaryl). In certain embodiments, A is —C(═O)NH(optionally substituted pyridinyl). In certain embodiments, A is —C(═O)NH(pyridinyl optionally substituted with halogen, —CN, or optionally substituted alkyl).

In certain embodiments, A is

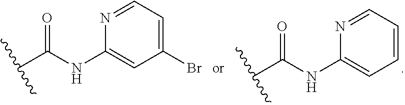 [SMILES: *C(=O)Nc1cc(Br)ccn1 ; *C(=O)Nc1ccccn1]

In certain embodiments, A is

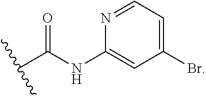 [SMILES: *C(=O)Nc1cc(Br)ccn1]

In certain embodiments, A is

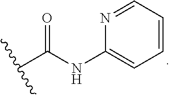 [SMILES: *C(=O)Nc1ccccn1]

In certain embodiments, A is —C(═O)(R⁵), wherein R⁵ is hydrogen, optionally substituted alkyl, optionally substituted carbocyclyl, optionally substituted aryl, optionally substituted heterocyclyl, or optionally substituted heteroaryl. In certain embodiments, A is —C(═O)H. In certain embodiments, A is —C(═O)(optionally substituted C₁₋₆ alkyl).

In certain embodiments, A is C(═O)OR⁵, wherein R⁵ is hydrogen, optionally substituted alkyl, optionally substituted carbocyclyl, optionally substituted aryl, optionally substituted heterocyclyl, or optionally substituted heteroaryl. In certain embodiments, A is —CN.

In certain embodiments, R² is hydrogen. In certain embodiments, R² is halogen (e.g., F, Cl, Br, or I). In certain embodiments, R² is F. In certain embodiments, R² is Cl. In certain embodiments, R² is Br. In certain embodiments, R² is I. In certain embodiments, R² is optionally substituted acyl (e.g., —C(=O)Me). In certain embodiments, R² is optionally substituted alkyl (e.g., substituted or unsubstituted C₁₋₆ alkyl). In certain embodiments, R² is substituted or unsubstituted methyl. In certain embodiments, R² is substituted or unsubstituted ethyl. In certain embodiments, R² is substituted or unsubstituted n-propyl. In certain embodiments, R² is unsubstituted isopropyl. In certain embodiments, R² is unsubstituted isopropyl. In certain embodiments, R² is alkenyl (e.g., substituted or unsubstituted C₂₋₆ alkenyl). In certain embodiments, R² is optionally substituted alkynyl (e.g., substituted or unsubstituted C₂₋₆ alkynyl). In certain embodiments, R² is optionally substituted carbocyclyl (e.g., substituted or unsubstituted, 3- to 7-membered, monocyclic carbocyclyl comprising zero, one, or two double bonds in the carbocyclic ring system). In certain embodiments, R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R² is optionally substituted, 5- to 10-membered monocyclic or bicyclic heterocyclic ring, wherein one or two atoms in the heterocyclic ring are independently nitrogen, oxygen, or sulfur. In certain embodiments, R² is

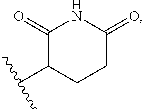 [SMILES: *C1CCC(=O)NC1=O]

which is optionally substituted. In certain embodiments, R² is

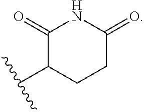 [SMILES: *C1CCC(=O)NC1=O]

In certain embodiments, R² is optionally substituted aryl (e.g., substituted or unsubstituted, 6- to 10-membered aryl). In certain embodiments, R² is optionally substituted benzyl. In certain embodiments, R² is optionally substituted phenyl. In certain embodiments, R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R² is —OR^{2A}, —N(R^{2B})₂, —SR^{2A}, as valency permits, wherein R^{2A} and R^{2B} are as defined herein. In certain embodiments, R² is —OR^{2A} (e.g., —OMe). In certain embodiments, R² is —N(R^{2B})₂ (e.g., —NH₂). In certain embodiments, R² is —SR^{2A} (e.g., —SMe). In certain embodiments, R² is unsubstituted isopropyl or

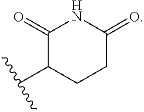 [SMILES: *C1CCC(=O)NC1=O]

In certain embodiments, at least one instance of R^{2A} is hydrogen. In certain embodiments, at least one instance of R^{2A} is substituted or unsubstituted acyl (e.g., —C(═O)Me). In certain embodiments, at least one instance of R^{2A} is optionally substituted alkyl (e.g., substituted or unsubstituted C₁₋₆ alkyl). In certain embodiments, at least one instance of R^{2A} is substituted or unsubstituted C₁₋₆ alkyl. In certain embodiments, at least one instance of R^{2A} is substituted or unsubstituted methyl. In certain embodiments, at least one instance of R^{2A} is substituted or unsubstituted ethyl. In certain embodiments, at least one instance of R^{2A} is substituted or unsubstituted n-propyl. In certain embodiments, at least one instance of R^{2A} is an oxygen protecting group when attached to an oxygen atom. In certain embodiments, at least one instance of R^{2A} is a sulfur protecting group when attached to a sulfur atom.

In certain embodiments, at least one instance of R^{2B} is hydrogen. In certain embodiments, two instances of R^{2B} are hydrogen. In certain embodiments, at least one instance of R^{2B} is substituted or unsubstituted acyl (e.g., —C(═O)Me). In certain embodiments, at least one instance of R^{2B} is optionally substituted alkyl (e.g., substituted or unsubstituted C₁₋₆ alkyl). In certain embodiments, at least one instance of R^{2B} is unsubstituted C₁₋₆ alkyl. In certain embodiments, two instances of R^{2B} are unsubstituted C₁₋₆ alkyl. In certain embodiments, two instances of R^{2B} are substituted or unsubstituted C₁₋₆ alkyl. In certain embodiments, at least one instance of R^{2B} is substituted or unsubstituted methyl. In certain embodiments, at least one instance of R^{2B} is substituted or unsubstituted ethyl. In certain embodiments, at least one instance of R^{2B} is substituted or unsubstituted propyl. In certain embodiments, at least one instance of R^{2B} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{2B} are taken together with their intervening atoms to form a substituted or unsubstituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or substituted or unsubstituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

In certain embodiments, R³ is hydrogen. In certain embodiments, R³ is halogen (e.g., F, Cl, Br, or I). In certain embodiments, R³ is F. In certain embodiments, R³ is Cl. In certain embodiments, R³ is Br. In certain embodiments, R³ is I. In certain embodiments, R³ is optionally substituted acyl (e.g., —C(═O)Me). In certain embodiments, R³ is optionally substituted alkyl (e.g., substituted or unsubstituted C₁₋₆ alkyl). In certain embodiments, R³ is optionally substituted C₁₋₆ alkyl. In certain embodiments, R³ is substituted or unsubstituted methyl. In certain embodiments, R³ is substituted methyl. In certain embodiments, R³ is unsubstituted methyl. In certain embodiments, R³ is substituted or unsubstituted ethyl. In certain embodiments, R³ is substituted or unsubstituted n-propyl. In certain embodiments, R³ is unsubstituted isopropyl. In certain embodiments, R³ is alkenyl (e.g., substituted or unsubstituted C₂₋₆ alkenyl). In certain embodiments, R³ is optionally substituted alkynyl (e.g., substituted or unsubstituted C₂₋₆ alkynyl). In certain embodiments, R³ is optionally substituted carbocyclyl (e.g., substituted or unsubstituted, 3- to 7-membered, monocyclic carbocyclyl comprising zero, one, or two double bonds in the carbocyclic ring system). In certain embodiments, R³ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R³ is optionally substituted, 5- to 10-membered monocyclic or bicyclic heterocyclic ring, wherein one or two atoms in the heterocyclic ring are independently nitrogen, oxygen, or sulfur. In certain embodiments, R³ is

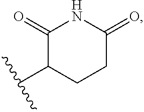 [SMILES: *C1CCC(=O)NC1=O]

which is optionally substituted. In certain embodiments, R³ is

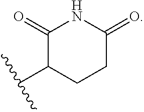 [SMILES: *C1CCC(=O)NC1=O]

In certain embodiments, R³ is optionally substituted aryl (e.g., substituted or unsubstituted, 6- to 10-membered aryl). In certain embodiments, R³ is optionally substituted benzyl. In certain embodiments, R³ is optionally substituted phenyl. In certain embodiments, R³ is unsubstituted phenyl. In certain embodiments, R³ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R³ is —OR^{2A} (e.g., —OMe). In certain embodiments, R³ is —N(R^{2B})₂ (e.g., —NH₂). In certain embodiments, R³ is —NH(optionally substituted alkyl). In certain embodiments, R³ is —NH (optionally substituted C₁₋₆ alkyl). In certain embodiments, R³ is

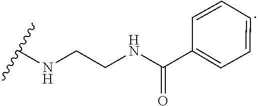 [SMILES: *NCCNC(=O)c1ccccc1]

In certain embodiments, R³ is —NH(CH₂)_{y}NHC(═O)(optionally substituted aryl), wherein y is 1, 2, 3, 4, 5, or 6. In certain embodiments, R³ is —NH(CH₂)_{y}NHC(═O)(optionally substituted phenyl), wherein y is 1, 2, 3, 4, 5, or 6. In certain embodiments, y is 1. In certain embodiments, y is 2. In certain embodiments, y is 3. In certain embodiments, y is 4. In certain embodiments, y is 5. In certain embodiments, y is 6. In certain embodiments, R³ is —SR^{2A} (e.g., —SMe). In certain embodiments, R³ is —OR^{2A}, —N(R^{2B})₂, or —SR^{2A}, as valency permits, wherein R^{2A} and R^{2B} are as defined herein. In certain embodiments, R³ is unsubstituted methyl, halogen, substituted or substituted phenyl, or

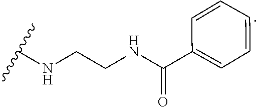 [SMILES: *NCCNC(=O)c1ccccc1]

In certain embodiments, R³ is unsubstituted methyl, chloro, unsubstituted phenyl, or

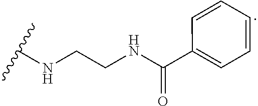 [SMILES: *NCCNC(=O)c1ccccc1]

In certain embodiments, R⁴ is hydrogen. In certain embodiments, R⁴ is halogen (F, Cl, Br, or I). In certain embodiments, R⁴ is F. In certain embodiments, R⁴ is Cl. In certain embodiments, R⁴ is Br. In certain embodiments, R⁴ is I. In certain embodiments, R⁴ is optionally substituted acyl (e.g., —C(═O)Me). In certain embodiments, R⁴ is optionally substituted alkyl (e.g., substituted or unsubstituted C₁₋₆ alkyl). In certain embodiments, R⁴ is optionally substituted C₁₋₆ alkyl. In certain embodiments, R⁴ is substituted or unsubstituted methyl. In certain embodiments, R⁴ is substituted methyl. In certain embodiments, R⁴ is unsubstituted methyl. In certain embodiments, R⁴ is substituted or unsubstituted ethyl. In certain embodiments, R⁴ is substituted or unsubstituted n-propyl. In certain embodiments, R⁴ is substituted or unsubstituted isopropyl. In certain embodiments, R⁴ is alkenyl (e.g., substituted or unsubstituted C₂₋₆ alkenyl). In certain embodiments, R⁴ is optionally substituted alkynyl (e.g., substituted or unsubstituted C₂₋₆ alkynyl). In certain embodiments, R⁴ is optionally substituted carbocyclyl (e.g., substituted or unsubstituted, 3- to 7-membered, monocyclic carbocyclyl comprising zero, one, or two double bonds in the carbocyclic ring system). In certain embodiments, R⁴ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R⁴ is optionally substituted, 5- to 10-membered monocyclic or bicyclic heterocyclic ring, wherein one or two atoms in the heterocyclic ring are independently nitrogen, oxygen, or sulfur. In certain embodiments, R⁴ is optionally substituted aryl (e.g., substituted or unsubstituted, 6- to 10-membered aryl). In certain embodiments, R⁴ is optionally substituted benzyl. In certain embodiments, R⁴ is optionally substituted phenyl. In certain embodiments, R⁴ is unsubstituted phenyl. In certain embodiments, R⁴ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R⁴ is —OR^{2A} (e.g., —OMe). In certain embodiments, R⁴ is —N(R^{2B})₂ (e.g., —NH₂). In certain embodiments, R⁴ is —NH(optionally substituted alkyl). In certain embodiments, R⁴ is —NH(optionally substituted C₁₋₆ alkyl). In certain embodiments, R⁴ is —SR²A (e.g., —SMe). In certain embodiments, R⁴ is —OR^{2A}, —N(R^{2B})₂, or —SR^{2A}, as valency permits, wherein R^{2A} and R^{2B} are as defined herein. In certain embodiments, R⁴ is —CN.

Formula (II) includes R⁷. The definition of R⁷ is as described herein, and is defined above.

In certain embodiments, R² is unsubstituted isopropyl or

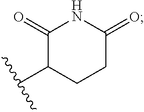 [SMILES: *C1CCC(=O)NC1=O]

unsubstituted methyl, halogen, substituted or substituted phenyl, or

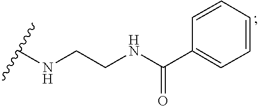 [SMILES: *NCCNC(=O)c1ccccc1]

R⁴ is hydrogen; R⁷ is hydrogen; and A is hydrogen, —C(═O)H, —C(═O)NH(optionally substituted heteroaryl), optionally substituted C₁₋₆ alkyl, or —CN. In certain embodiments, R² is unsubstituted isopropyl or

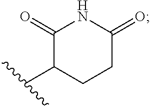 [SMILES: *C1CCC(=O)NC1=O]

R³ is unsubstituted methyl, chloro, unsubstituted phenyl, or

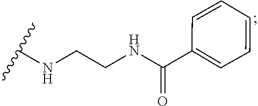 [SMILES: *NCCNC(=O)c1ccccc1]

R⁴ is hydrogen; R⁷ is hydrogen; A is hydrogen, —C(═O)H,

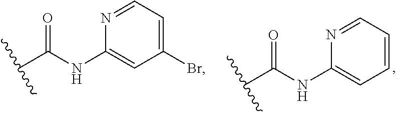 [SMILES: *C(=O)Nc1cc(Br)ccn1 ; *C(=O)Nc1ccccn1]

unsubstituted methyl, or —CN.

In certain embodiments, the compound of Formula (II) is represented by any one of structures:

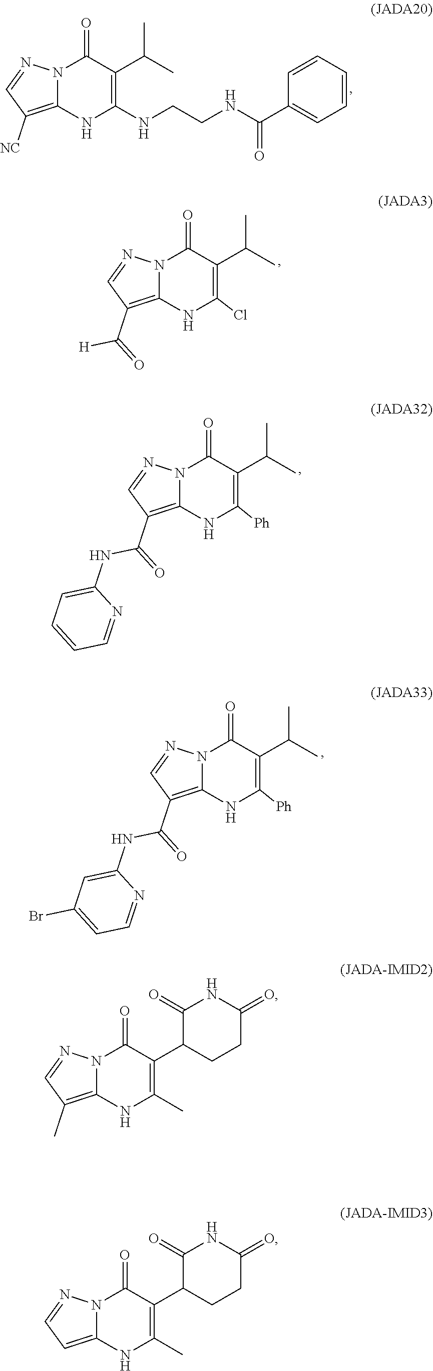 [SMILES: CC(C)c1c(NCCNC(=O)c2ccccc2)[nH]c2c(C#N)cnn2c1=O ; CC(C)c1c(Cl)[nH]c2c(C=O)cnn2c1=O ; CC(C)c1c(-c2ccccc2)[nH]c2c(C(=O)Nc3ccccn3)cnn2c1=O ; CC(C)c1c(-c2ccccc2)[nH]c2c(C(=O)Nc3cc(Br)ccn3)cnn2c1=O ; Cc1[nH]c2c(C)cnn2c(=O)c1C1CCC(=O)NC1=O ; Cc1[nH]c2ccnn2c(=O)c1C1CCC(=O)NC1=O]

or a pharmaceutically acceptable salt, solvate, hydrate, polymorph, co-crystal, tautomer, stereoisomer, isotopically labeled derivative, or prodrug thereof.

Other exemplary compounds include, but are not limited to:

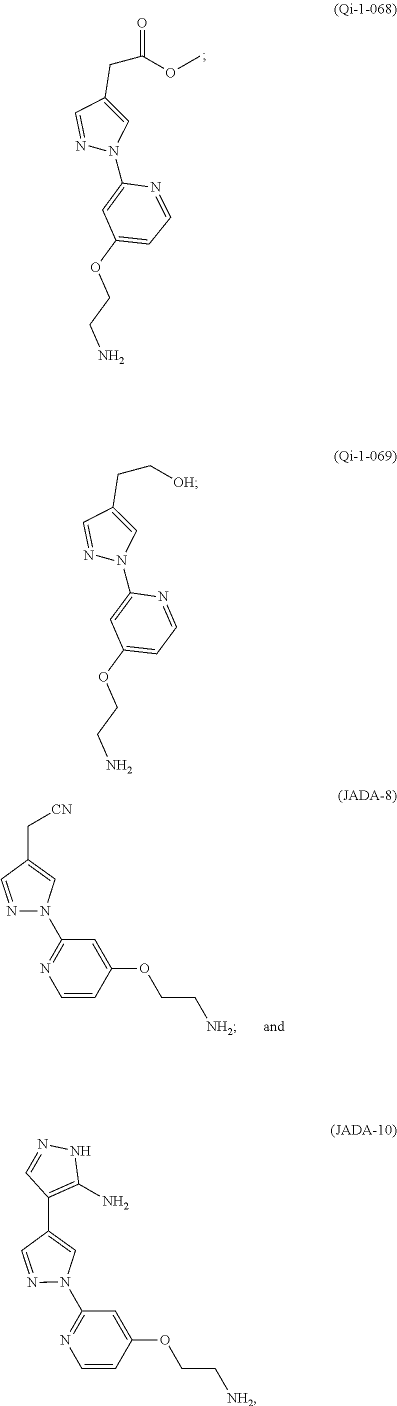 [SMILES: COC(=O)Cc1cnn(-c2cc(OCCN)ccn2)c1 ; NCCOc1ccnc(-n2cc(CCO)cn2)c1 ; N#CCc1cnn(-c2cc(OCCN)ccn2)c1 ; NCCOc1ccnc(-n2cc(-c3cn[nH]c3N)cn2)c1]

and pharmaceutically acceptable salts, solvates, hydrates, polymorphs, co-crystals, tautomers, stereoisomers, isotopically labeled derivatives, and prodrugs thereof.

In certain embodiments, the compound of Formula (I) is a compound provided in any one of the Examples below. In certain embodiments, the compound of Formula (II) is a compound provided in any one of the Examples below.

In certain embodiments, a compound described herein is a compound of Formula (I) or (II), Qi-1-068, Qi-1-069, JADA-8, JADA-10 or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I) or (II), or a pharmaceutically acceptable salt thereof.

Certain compounds described herein induce the degradation of a target transcription factor. In certain embodiments, the compounds described herein degrade a target transcription factor. In certain embodiments, the target transcription factor is IKZF1. In certain embodiments, the target transcription factor is IKZF3. In certain embodiments, the compounds described herein bind to an E3 ubiquitin ligase and induce degradation of a target transcription factor (e.g., IKZF1, IKZF3). In certain embodiments, the compounds described herein bind to E3 ubiquitin ligase Cereblon and induce degradation of a target transcription factor (e.g., IKZF1, IKZF3). In certain embodiments, the compounds described herein are binders or ligands of Cereblon. In certain embodiments, the compounds described herein are binders of Cereblon. In certain embodiments, the compounds described herein are ligands of Cereblon. In certain embodiments, the compounds described herein are modulators, binders, inhibitors, or ligands of a Cereblon variant. In certain embodiments, the compounds described herein are modulators, binders, inhibitors, or ligands of a Cereblon isoform.

The binding affinity of a compound described herein to Cereblon may be measured by the dissociation constant (K_{d}) value of an adduct of the compound and Cereblon using methods known in the art (e.g., isothermal titration calorimetry (ITC)). In certain embodiments, the K_{d} value of the adduct is not more than about 100 μM, not more than about 10 μM, not more than about 1 μM, not more than about 100 nM, not more than about 10 nM, or not more than about 1 nM.

In certain embodiments, the compound of Formula (I) or (II) induces the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of the target transcription factor (e.g., IKZF1, IKZF3) at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) or (II) increases the rate of degradation of the target transcription factor (e.g., IKZF1, IKZF3) up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

It is expected that the compounds described herein may be useful in treating and/or preventing diseases associated with a transcription factor (e.g., IKZF1, IKZF3). It is known in the art that the transcription factors IKZF1 or IKZF3 are implicated in various diseases including proliferative diseases. Therefore, the compounds described herein are expected to be useful in treating and/or preventing diseases (e.g., proliferative diseases (e.g., cancers)).

### Pharmaceutical Compositions, Kits, and Administration

The present disclosure provides pharmaceutical compositions comprising a compound of Formula (I) or (II), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I) or (II), or a pharmaceutically acceptable salt thereof, and a pharmaceutically acceptable excipient.

In certain embodiments, the compound of Formula (I) or (II) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for reducing the risk of developing a disease (e.g., proliferative disease, such as cancer) in a subject in need thereof.

In certain embodiments, the effective amount is an amount effective for inducing the degradation of at least about 10%, at least about 20%, at least about 30%, at least about 40%, at least about 50%, at least about 60%, at least about 70%, at least about 80%, at least about 90%, at least about 95%, at least about 98%, or at least about 99% of the transcription factor IKZF1 or IKZF3 in a cell. In certain embodiments, the effective amount is an amount effective for inducing the degradation of at least about 10%, at least about 20%, at least about 30%, at least about 40%, at least about 50%, of the transcription factor IKZF1 or IKZF3 in a cell. In certain embodiments, the effective amount is an amount effective for inducing the degradation of at least about 50%, at least about 60%, at least about 70%, at least about 80%, at least about 90%, at least about 95%, at least about 98%, or at least about 99% of the transcription factor IKZF1 or IKZF3 in a cell. In certain embodiments, the effective amount is an amount effective for inducing the degradation of transcription factor IKZF1 or IKZF3 in a cell by a range between a percentage described in this paragraph and another percentage described in this paragraph, inclusive.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj ® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, German® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample (e.g., tissue or cell), any two doses of the multiple doses include different or substantially the same amounts of a compound described herein. In certain embodiments, when multiple doses are administered to a subject or applied to a biological sample (e.g., tissue or cell), the frequency of administering the multiple doses to the subject or applying the multiple doses to the biological sample (e.g., tissue or cell) is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample (e.g., tissue or cell) is one dose per day. In certain embodiments, the frequency of administering the multiple doses to the subject or applying the multiple doses to the biological sample (e.g., tissue or cell) is two doses per day. In certain embodiments, the frequency of administering the multiple doses to the subject or applying the multiple doses to the biological sample (e.g., tissue or cell) is three doses per day. In certain embodiments, when multiple doses are administered to a subject or applied to a biological sample (e.g., tissue or cell), the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease) in a subject in need thereof. In certain embodiments, the kits are useful for preventing a disease (e.g., proliferative disease) in a subject in need thereof.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease) in a subject in need thereof. In certain embodiments, the kits and instructions provide for preventing a disease (e.g., proliferative disease) in a subject in need thereof. In certain embodiments, the kits and instructions provide for inducing the degradation of transcription factor IKZF1 or IKZF3 in a subject or biological sample (e.g., tissue or cell). A kit described herein may include one or more additional pharmaceutical agents described herein as a separate composition.

### Methods of Treatment and Uses

The compounds described herein are capable of binding (e.g., reversibly binding or irreversibly binding) an E3 ubiquitin ligase (e.g., Cereblon) and a transcription factor (e.g., IKZF1, IKZF3) and inducing the degradation of the transcription factor. The present disclosure thus also provides methods of inducing the degradation of a transcription factor (e.g., IKZF1, IKZF3) in a subject or biological sample (e.g., tissue or cell). The present disclosure further provides methods for the treatment of proliferative diseases in a subject in need thereof

In certain embodiments, the application provides a method of binding an E3 ubiquitin ligase (e.g., Cereblon) and promoting the degradation of a transcription factor (e.g., IKZF 1 or IKZF3). In another aspect, the present disclosure provides methods of inducing the degradation of a transcription factor (e.g., IKZF1 or IKZF3) in a subject in need thereof, the methods comprise administering to the subject an effective amount of a compound or pharmaceutical composition described herein. In another aspect, the present disclosure provides methods of inducing the degradation of a transcription factor (e.g., IKZF1 or IKZF3), and thereby degrading transcription factor SALL4 in a subject in need thereof, the methods comprise administering to the subject an effective amount of a compound or pharmaceutical composition described herein. In another aspect, the present disclosure provides methods of inducing the degradation of a transcription factor (e.g., IKZF1 or IKZF3) in a biological sample (e.g., tissue or cell), the methods comprise contacting the biological sample (e.g., tissue or cell) with an effective amount of a compound or pharmaceutical composition described herein. In another aspect, the present disclosure provides methods of inducing the degradation of a transcription factor (e.g., IKZF1 or IKZF3), and thereby degrading transcription factor SALL4 in a biological sample (e.g., tissue or cell), the methods comprise contacting the biological sample (e.g., tissue or cell) with an effective amount of a compound or pharmaceutical composition described herein.

In certain embodiments, the application provides a method of binding an E3 ubiquitin ligase (e.g., Cereblon) and a transcription factor (e.g., IKZF1, IKZF3), and inducing the degradation of the transcription factor. In certain embodiments, the transcription factor that is bound is IKZF1. In certain embodiments, the transcription factor that is bound is IKZF3. In certain embodiments, the transcription factor that is bound and degraded is IKZF1. In certain embodiments, the transcription factor that is bound and degraded is IKZF3. In certain embodiments, the transcription factor that is bound is IKZF1 and/or IKZF3, thereby degrading SALL4.

The present disclosure also provides a compound of Formulae (I) or (II), or a pharmaceutically acceptable salt, co-crystal, tautomer, stereoisomer, solvate, hydrate, polymorph, isotopically enriched derivative, or prodrug, or composition thereof, for use in the treatment of a disease, such as proliferative disease, in a subject in need thereof. Exemplary proliferative diseases which may be treated include diseases associated with a target transcription factor (e.g., IKZF1, IKZF3), e.g., cancer. In certain embodiments, the cancer is a carcinoma. In certain embodiments, the cancer is lung cancer, breast cancer, liver cancer, pancreatic cancer, gastric cancer, ovarian cancer, colon cancer, or colorectal cancer. In other embodiments, the cancer is multiple myeloma or leukemia such as acute myeloid leukemia (AML).

In certain embodiments, the lung cancer is non-small cell lung cancer. In certain embodiments, the cancer is liver cancer. In certain embodiments, the cancer is multiple myeloma. In certain embodiments, the cancer is leukemia. In certain embodiments, the leukemia is acute myeloid leukemia (AML).

The present disclosure also provides uses of a compound of Formulae (I) or (II), or a pharmaceutically acceptable salt, co-crystal, tautomer, stereoisomer, solvate, hydrate, polymorph, isotopically enriched derivative, or prodrug, or composition thereof, in the manufacture of a medicament for the treatment of a disease, such as a proliferative disease, in a subject in need thereof.

In certain embodiments, the methods of the disclosure comprise administering to the subject an effective amount of a compound of Formula (I) or (II), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formula (I) or (II), or at different times than the compound of Formula (I) or (II). For example, the compound of Formula (I) or (II) and any additional pharmaceutical agent(s) may be on the same dosing schedule or different dosing schedules. All or some doses of the compound of Formula (I) or (II) may be administered before all or some doses of an additional pharmaceutical agent, after all or some does an additional pharmaceutical agent, within a dosing schedule of an additional pharmaceutical agent, or a combination thereof. The timing of administration of the compound of Formula (I) or (II) and additional pharmaceutical agents may be different for different additional pharmaceutical agents.

In certain embodiments, the additional pharmaceutical agent comprises an agent useful in the treatment of diseases, such as proliferative diseases, inflammatory diseases, autoimmune diseases, genetic diseases, hematological diseases, neurological diseases, painful conditions, psychiatric disorders, and metabolic disorders in a subject in need thereof. In certain embodiments, the additional pharmaceutical agent is useful in the treatment of a proliferative disease. In certain embodiments, the additional pharmaceutical agent is useful in the treatment of an inflammatory disease. In certain embodiments, the additional pharmaceutical agent is useful in the treatment of proliferative diseases, inflammatory diseases, autoimmune diseases, genetic diseases, hematological diseases, neurological diseases, painful conditions, psychiatric disorders, or metabolic disorders. In certain embodiments, the additional pharmaceutical agent is useful in the treatment of multiple myeloma. In certain embodiments, the additional pharmaceutical agent is useful in the treatment of leukemia. In certain embodiments, the additional pharmaceutical agent is useful in the treatment of lymphoma. In certain embodiments, the additional pharmaceutical agent is useful in the treatment of a non-Hodgkin's lymphoma.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inducing the degradation of a target protein, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or biological sample (e.g., tissue, cell). It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, inflammatory disease, autoimmune disease, genetic disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, cytotoxic chemotherapeutic agents, epigenetic modifiers, glucocorticoids, immunotherapeutic agents, anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is abiraterone acetate (e.g., ZYTIGA®), a combination of Adriamycin® (a.k.a. Doxorubicin), Bleomycin, Vinblastine, and Dacarbazine (ABVD) , a combination of Adriamycin®, Bleomycin, Vinblastine, and Etoposide (ABVE), a combination of Adriamycin®, Bleomycin, Vinblastine, Etoposide, Prednisone, and Cyclophosphamide (ABVE-PC), a combination of Adriamycin® and Cyclophosphamide (AC), a combination of Adriamycin®, Cyclophosphamide, followed by Taxol® (AC-T), a combination of Cytarabine (Ara-C), Daunorubicin, and Etoposide (ADE), ado-trastuzumab emtansine (e.g., KADCYLA®), afatinib dimaleate (e.g., GILOTRIF®), aldesleukin (e.g., PROLEUKIN®), alemtuzumab (e.g., CAMPATH®), anastrozole (e.g., ARIMIDEX®), arsenic trioxide (e.g., TRISENOX®), asparaginase erwinia chrysanthemi (e.g., ERWINAZE®), axitinib (e.g., INLYTAO), azacitidine (e.g., MYLOSAR®, VIDAZA®), a combination of Bleomycin, Etoposide, Doxorubicin, Clophosphamide, Vincristine, Procarbazine, and Prednisone (BEACOPP), belinostat (e.g., BELEODAQ®), bendamustine hydrochloride (e.g., TREANDA®), a combination of Bleomycin, etoposide, and platinum (BEP), bevacizumab (e.g., AVASTIN®), bicalutamide (e.g., CASODEX®), bleomycin (e.g., BLENOXANE®), blinatumomab (e.g., BLINCYTO®), bortezomib (e.g., VELCADE®), bosutinib (e.g., BOSULIF®), brentuximab vedotin (e.g., ADCETRIS®), busulfan (e.g., BUSULFEX®, MYLERAN®), cabazitaxel (e.g., JEVTANA®), cabozantinib-s-malate (e.g., COMETRIQ®), CAF, capecitabine (e.g., XELODA®), a combination of capecitabine and oxaliplatin (CAPOX), carboplatin (e.g., PARAPLATIN®), carboplatin-taxol, carfilzomib (e.g., KYPROLIS®), carmustine (e.g., BECENUM®, BICNU®, CARMUBRIS®), carmustine implant (e.g., GLIADEL® WAFER, GLIADEL®), ceritinib (e.g., ZYKADIA®), cetuximab (e.g., ERBITUX®), chlorambucil (e.g., LEUKERAN®), chlorambucil-prednisone, a combination of Cyclophosphamide, Hydroxydaunorubicin, Oncovin®, and Prednisone (CHOP), cisplatin (e.g., PLATINOL®, PLATINOL®-AQ), clofarabine (e.g., CLOFAREX®, CLOLAR®), a combination of Cyclophosphamide, Methotrexate, and Fluorouracil (CMF), a combination of Cyclophosphamide, Oncovin, Procarbazine, and Prednisone (COPP), COPP-alternating with doxorubicin, bleomycin and vinblastine (ABV), crizotinib (e.g., XALKORI®), a combination of Cyclophosphamide, Vincristine, and Prednisone (CVP), cyclophosphamide (e.g., CLAFEN®, CYTOXAN®), cytarabine (e.g., CYTOSAR-U, TARABINE PFS), dabrafenib (e.g., TAFINLAR®), dacarbazine (e.g., DTIC-DOME®), dactinomycin (e.g., COSMEGEN®), dasatinib (e.g., SPRYCEL®), daunorubicin hydrochloride (e.g., CERUBIDINE®), decitabine (e.g., DACOGEN®), degarelix, denileukin diftitox (e.g., ONTAK®), denosumab (e.g., PROLIA®, XGEVA®), Dinutuximab (e.g., UNITUXIN®), docetaxel (e.g., TAXOTERE®), doxorubicin hydrochloride (e.g., ADRIAMYCIN® PFS, ADRIAMYCIN® RDF), doxorubicin hydrochloride liposome (e.g., DOXIL®, DOX-SL®, EVACET®, LIPODOX®), enzalutamide (e.g., XTANDI®), epirubicin hydrochloride (e.g., ELLENCE®), a combination of Etoposide, Prednisone, Oncovin®, Cyclophosphamide, and Hydroxydaunorubicin (EPOCH), erlotinib hydrochloride (e.g., TARCEVA®), etoposide (e.g., TOPOSAR®, VEPESID®), etoposide phosphate (e.g., ETOPOPHOS®), everolimus (e.g., AFINITOR DISPERZ®, AFINITOR®), exemestane (e.g., AROMASIN®), a combination of 5-Fluorouracil, Epirubicin, and Cyclophosphamide (FEC), fludarabine phosphate (e.g., FLUDARA®), fluorouracil (e.g., ADRUCIL®, EFUDEX®, FLUOROPLEX®), FOLFIRI®, FOLFIRI®-BEVACIZUMAB, FOLFIRI®-CETUXIMAB, a combination of bolus and 5-Fluorouracil, Leucovorin, Irinotecan and Oxaliplatin (FOLFIRINOX), a combination of 5-Fluorouracil, Leucovorin, Oxaliplatin (FOLFOX), a combination of 5-Fluorouracil and Leucovorin (FU-LV), fulvestrant (e.g., FASLODEX®), gefitinib (e.g., IRESSA®), gemcitabine hydrochloride (e.g., GEMZAR®), gemcitabine-cisplatin, gemcitabine-oxaliplatin, goserelin acetate (e.g., ZOLADEX®), hyperfractionated cyclophosphamide, vincristine , Adriamycin®, and dexamethasone (Hyper-CVAD), ibritumomab tiuxetan (e.g., ZEVALIN®), ibrutinib (e.g., IMBRUVICA®), a combination of Ifosfamide, Carboplatin, and Etoposide (ICE), idelalisib (e.g., ZYDELIG®), ifosfamide (e.g., CYFOS®, IFEX®-), imatinib mesylate (e.g., GLEEVEC®), imiquimod (e.g., ALDARA®), ipilimumab (e.g., YERVOY®), irinotecan hydrochloride (e.g., CAMPTOSAR®), ixabepilone (e.g., IXEMPRA®), lanreotide acetate (e.g., SOMATULINE® DEPOT), lapatinib ditosylate (e.g., TYKERB®), lenalidomide (e.g., REVLIMID®), lenvatinib (e.g., LENVIMA®), letrozole (e.g., FEMARA®), leucovorin calcium (e.g., WELLCOVORIN®), leuprolide acetate (e.g., LUPRON DEPOT®, LUPRON DEPOT®-3 MONTH, LUPRON DEPOT®-4 MONTH, LUPRON DEPOT-PED®, LUPRON®, VIADUR®), liposomal cytarabine (e.g., DEPOCYT®), lomustine (e.g., CEENU®), mechlorethamine hydrochloride (e.g., MUSTARGEN®), megestrol acetate (e.g., MEGACE®), mercaptopurine (e.g., PURINETHOL®, PURIXAN®), methotrexate (e.g., ABITREXATE®, FOLEX PFS®, FOLEX®, METHOTREXATE LPF®, MEXATE®, MEXATE-AQ), mitomycin c (e.g., MITOZYTREX®, MUTAMYCIN®), mitoxantrone hydrochloride, a combination of Mechlorethamine, Oncovin®, Procarbazine, and Prednisone (MOPP), nelarabine (e.g., ARRANON®), nilotinib (e.g., TASIGNA®), nivolumab (e.g., OPDIVO®), obinutuzumab (e.g., GAZYVA®), OEPA, ofatumumab (e.g., ARZERRA®), a combination of Oxaliplatin, 5-Fluorouracil, and Folinic acid (OFF), olaparib (e.g., LYNPARZA®), omacetaxine mepesuccinate (e.g., SYNRIBO®), a combination of Oncovin®, Procarbazine, Prednisone, and Adriamycin® (OPPA), oxaliplatin (e.g., ELOXATIN®), paclitaxel (e.g., TAXOL®), paclitaxel albumin-stabilized nanoparticle formulation (e.g., ABRAXANE®), PAD, palbociclib (e.g., IBRANCE®), pamidronate disodium (e.g., AREDIA®), panitumumab (e.g., VECTIBIX®), panobinostat (e.g., FARYDAK®), pazopanib hydrochloride (e.g., VOTRIENT®), pegaspargase (e.g., ONCASPAR®), peginterferon alfa-2b (e.g., PEGINTRON®), SYLATRON™), pembrolizumab (e.g., KEYTRUDA®), pemetrexed disodium (e.g., ALIMTA®), pertuzumab (e.g., PERJETA®), plerixafor (e.g., MOZOBIL®), pomalidomide (e.g., POMALYST®), ponatinib hydrochloride (e.g., ICLUSIG®), pralatrexate (e.g., FOLOTYN®), prednisone, procarbazine hydrochloride (e.g., MATULANE®), radium 223 dichloride (e.g., XOFIGO®), raloxifene hydrochloride (e.g., EVISTA®, KEOXIFENE®), ramucirumab (e.g., CYRAMZA®), a combination of Rituximab, Cyclophosphamide, Hydroxydaunorubicin, Oncovin®, and Prednisone (R-CHOP), recombinant HPV bivalent vaccine (e.g., CERVARIX®), recombinant human papillomavirus (e.g., HPV) nonavalent vaccine (e.g., GARDASIL® 9), recombinant human papillomavirus (e.g., HPV) quadrivalent vaccine (e.g., GARDASIL®), recombinant interferon alfa-2b (e.g., INTRON® A), regorafenib (e.g., STIVARGA®), rituximab (e.g., RITUXAN®), romidepsin (e.g., ISTODAX®), ruxolitinib phosphate (e.g., JAKAFI®), siltuximab (e.g., SYLVANT®), sipuleucel-t (e.g., PROVENGE®), sorafenib tosylate (e.g., NEXAVAR®), STANFORD V, sunitinib malate (e.g., SUTENT®), a combination of Taxotere®, Adriamycin®, and Cyclophosphamide (TAC), tamoxifen citrate (e.g., NOLVADEX®, NOVALDEX®), temozolomide (e.g., METHAZOLASTONE®, TEMODAR®), temsirolimus (e.g., TORISEL®), thalidomide (e.g., SYNOVIR®, THALOMID®), thiotepa, topotecan hydrochloride (e.g., HYCAMTIN®), toremifene (e.g., FARESTON®), tositumomab and iodine I 131 tositumomab (e.g., BEXXAR®), TPF, trametinib (e.g., MEKINIST®), trastuzumab (e.g., HERCEPTIN®), a combination of Vincristine sulfate, Adriamycin®, Methotrexate, and Prednisone (VAMP), vandetanib (e.g., CAPRELSA®), a combination of Velban®, Ifosfamide , and Platinol® (VEIP), vemurafenib (e.g., ZELBORAF®), vinblastine sulfate (e.g., VELBAN®, VELSAR®), vincristine sulfate (e.g., VINCASAR PFS®), vincristine sulfate liposome (e.g., MARQIB®), vinorelbine tartrate (e.g., NAVELBINE), vismodegib (e.g., ERIVEDGE®), vorinostat (e.g., ZOLINZA®), XELIRI, a combination of Xeloda® and Oxaliplatin (XELOX), ziv-aflibercept (e.g., ZALTRAP®), or zoledronic acid (e.g., ZOMETA®). In certain embodiments, the additional pharmaceutical agent is ENMD-2076, PCI-32765, AC220, dovitinib lactate (e.g., TKI258, CHIR-258), BIBW 2992 (e.g., TOVOKTm), SGX523, PF-04217903, PF-02341066, PF-299804, BMS-777607, ABT-869, MP470, BIBF 1120 (e.g., VARGATEF®), AP24534, JNJ-26483327, MGCD265, DCC-2036, BMS-690154, CEP-11981, tivozanib (e.g., AV-951), OSI-930, MM-121, XL-184, XL-647, and/or XL228), proteasome inhibitors (e.g., bortezomib (e.g., Velcade)), mTOR inhibitors (e.g., rapamycin, temsirolimus (e.g., CCI-779), everolimus (e.g., RAD-001), ridaforolimus, AP23573 (e.g., Ariad), AZD8055, BEZ235, BGT226, XL765, PF-4691502, GDC0980, SF1126, and OSI-027), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomy cin aminopterin, and hexamethyl melamine, or a combination thereof. In certain embodiments, the additional pharmaceutical agent is a cytotoxic chemotherapy (e.g., cytotoxic chemotherapeutic agent (e.g., gemcitabine, cytarabine, daunorubicin, doxorubicin, vincristine, 1-asparaginase, cyclophosphamide, or etoposide)). In certain embodiments, the additional pharmaceutical agent is an epigenetic modifier, such as azacitidine or romidepsin. In certain embodiments, the additional pharmaceutical agent is ruxolitinib, BBT594, CHZ868, CYT387, or BMS911543. In certain embodiments, the additional pharmaceutical agent is an inhibitor of a tyrosine kinase. In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-4-(((5′-chloro-2′-(((1R,4R)-4-4-(R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. In certain embodiments, the additional pharmaceutical agent is a binder or inhibitor of a kinase (e.g., CDK). In certain embodiments, the additional pharmaceutical agent is an antibody or a fragment thereof (e.g., monoclonal antibody). In certain embodiments, the additional pharmaceutical agent is a tyrosine kinase inhibitor. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the additional pharmaceutical agent is a glucocorticoid (e.g., cortisol, cortisone, prednisone, methylprednisolone, dexamethasone, betamethasone, triamcinolone, fludrocortisone acetate, or deoxycorticosterone acetate). In certain embodiments, the additional therapy is an immunotherapy (e.g., an immunotherapeutic monoclonal antibody). In certain embodiments, the additional pharmaceutical agent is an immunomodulator. In certain embodiments, the additional pharmaceutical agent is an immune checkpoint inhibitor. In certain embodiments, the additional pharmaceutical agent is a programmed cell death 1 protein (PD-1) inhibitor. In certain embodiments, the additional pharmaceutical agent is a programmed cell death 1 protein ligand 1 (PD-L1) inhibitor. In certain embodiments, the additional pharmaceutical agent is a cytotoxic T-lymphocyte-associated protein 4 (CTLA-4) inhibitor. In certain embodiments, the additional pharmaceutical agent is a T-cell immunoglobulin domain and mucin domain 3 (TIM3) inhibitor, lymphocyte activation gene-3 (LAG3) inhibitor, V-set domain-containing T-cell activation inhibitor 1 (VTCN1 or B7-H4) inhibitor, cluster of differentiation 276 (CD276 or B7-H3) inhibitor, B and T lymphocyte attenuator (BTLA) inhibitor, galectin-9 (GAL9) inhibitor, checkpoint kinase 1 (Chk1) inhibitor, adenosine A2A receptor (A2AR) inhibitor, indoleamine 2,3-dioxygenase (IDO) inhibitor, killer-cell immunoglobulin-like receptor (KIR) inhibitor, or V-domain Ig suppressor of T cell activation (VISTA) inhibitor. In certain embodiments, the PD-1 inhibitor is nivolumab, pidilizumab, pembrolizumab, MEDI-0680, REGN2810, or AMP-224. In certain embodiments, the PD-L1 inhibitor is atezolizumab, durvalumab, BMS-936559, avelumab, or CA-170. In certain embodiments, the CTLA-4 inhibitor is ipilimumab or tremelimumab. In certain embodiments, the additional pharmaceutical agent is an aromatase inhibitor. In certain embodiments, the additional pharmaceutical agent is a PI3K inhibitor. In certain embodiments, the additional pharmaceutical agent is an mTOR inhibitor. In certain embodiments, the additional pharmaceutical agent is an endocrine therapy. In certain embodiments, the additional pharmaceutical agent is an epigenetic target inhibitor (e.g., bromodomain inhibitor, DNA methylation inhibitor, histone acetyl transferase inhibitor, histone deacetylase inhibitor, protein methyltransferase inhibitor, histone methylation inhibitor). In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. In certain embodiments, the compounds described herein or pharmaceutical compositions can be administered in combination with chemotherapy. In certain embodiments, the compounds described herein or pharmaceutical compositions can be administered in combination with immunotherapy. In certain embodiments, the compounds described herein or pharmaceutical compositions can be administered in combination with chemotherapy or immunotherapy. In certain embodiments, the compounds described herein or pharmaceutical compositions can be administered in combination with proteasome inhibitors, monoclonal antibodies, and/or immune checkpoint blockers.

In another aspect, the present disclosure provides methods for inducing the degradation of a transcription factor (e.g., IKZF1 or IKZF3), the method comprising administering to the subject a compound of Formula (I) or (II), or a pharmaceutically acceptable salt, co-crystal, tautomer, stereoisomer, solvate, hydrate, polymorph, isotopically enriched derivative, or prodrug, or composition thereof. In certain embodiments, the additional pharmaceutical agent is an epigenetic target inhibitor (e.g., bromodomain inhibitor, DNA methylation inhibitor, histone acetyl transferase inhibitor, histone deacetylase inhibitor, protein methyltransferase inhibitor, histone methylation inhibitor).

In another aspect, the present disclosure provides methods for binding an E3 ubiquitin ligase and promoting the degradation of a transcription factor (e.g., IKZF 1, IKZF3), the method comprising administering to the subject a compound of Formula (I) or (II), or a pharmaceutically acceptable salt, co-crystal, tautomer, stereoisomer, solvate, hydrate, polymorph, isotopically enriched derivative, or prodrug, or composition thereof. In one aspect, the present disclosure provides methods for inducing the degradation of a transcription factor (e.g., IKZF1, IKZF3).

In still another aspect, the present disclosure provides the pharmaceutical compositions described herein for use in binding an E3 ubiquitin ligase and a transcription factor (e.g., IKZF1, IKZF3). The present disclosure also provides the pharmaceutical compositions described herein for use in promoting the degradation of the transcription factor (e.g., IKZF1, IKZF3); treating and/or preventing proliferative diseases.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

### Example 1

### Exemplary E3 Ligase Binding Compounds and Uses Thereof

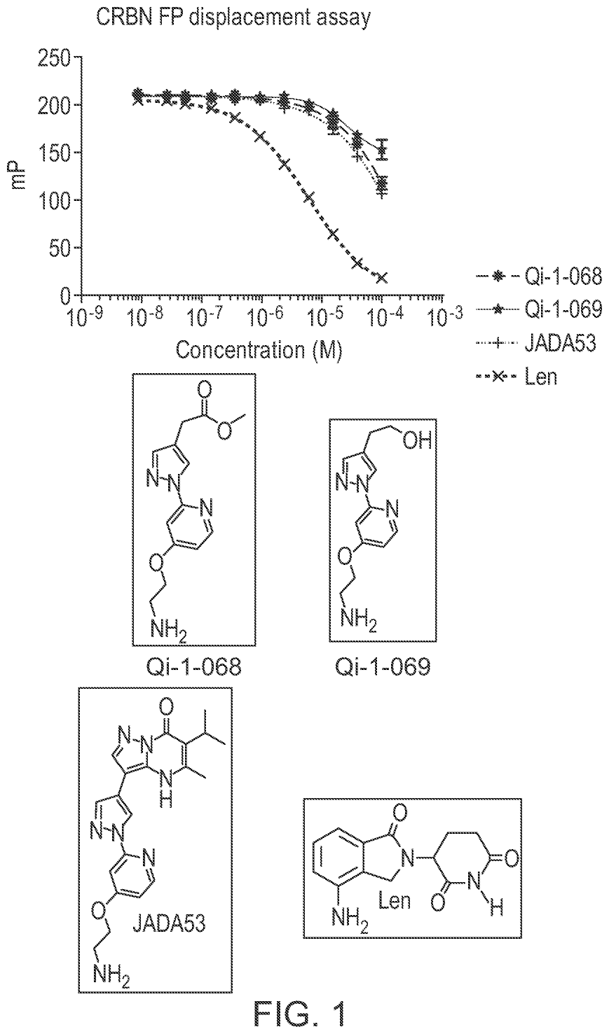

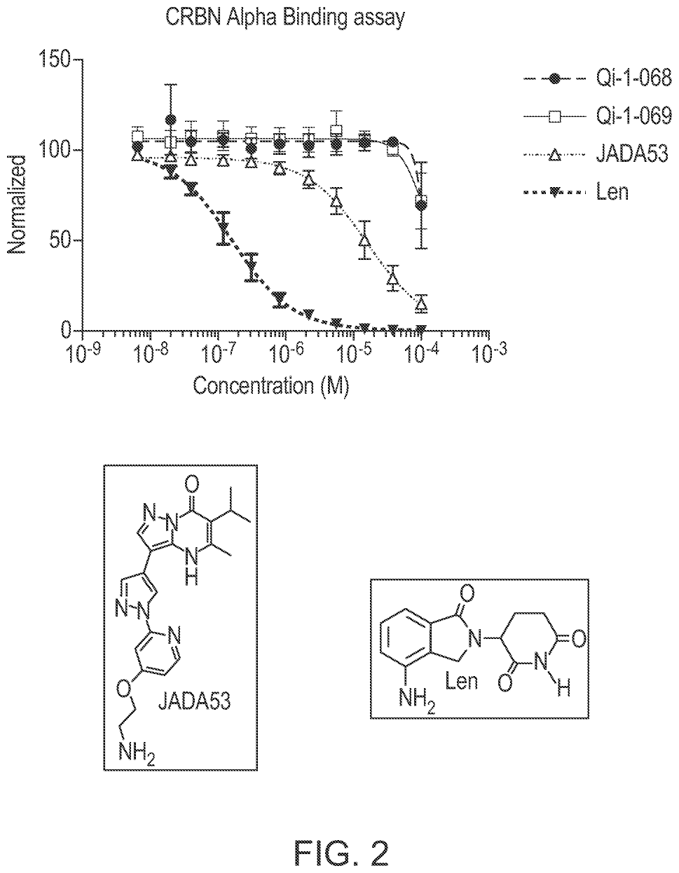

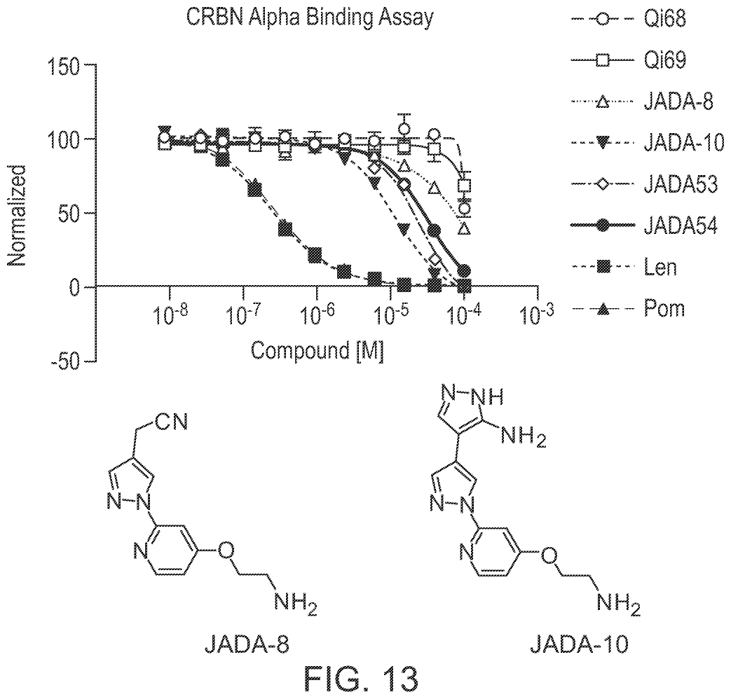

Results of the CRBN Fluorescence Polarization (FP) displacement assay and CRBN alpha binding assay of tested exemplary compounds are shown in FIG. 1 and FIGS. 2 and 13, respectively. Inventive compound JADA53 as well as the intermediate structures, e.g., JADA-8 and JADA-10, showed that the core of CRBN binding is the pyridine-imidazole group. Compound JADA-8 is the core for the binding.

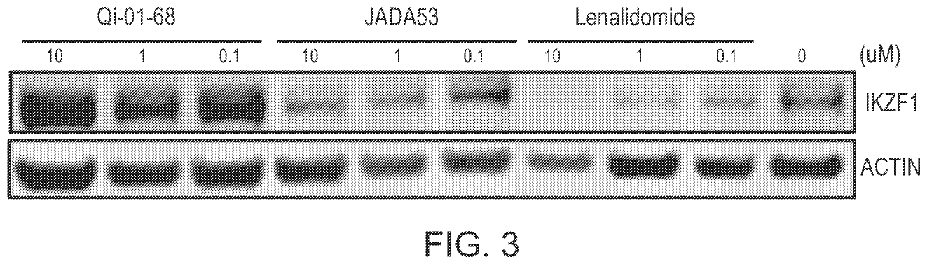

The results of an assay for CRBN binding (and degradation of IKZF or actin) upon treatment for 24 hours with exemplary compounds Qi-01-68, JADA53, and positive control lenalidomide (at the indicated concentrations in μM) in 293T WT cells (homo sapiens kidney cell line; wild type) are shown in FIG. 3. Exemplary compound 53 (JADA53) showed good binding of target CRBN in the cell, where binding with CRBN in the cell degraded IKZF. According to the CRBN FP displacement assay (FIG. 1), exemplary compound Qi-01-68 had the same binding activity as JADA53, however, it did not degrade IKZF1 in the cell (FIG. 3).

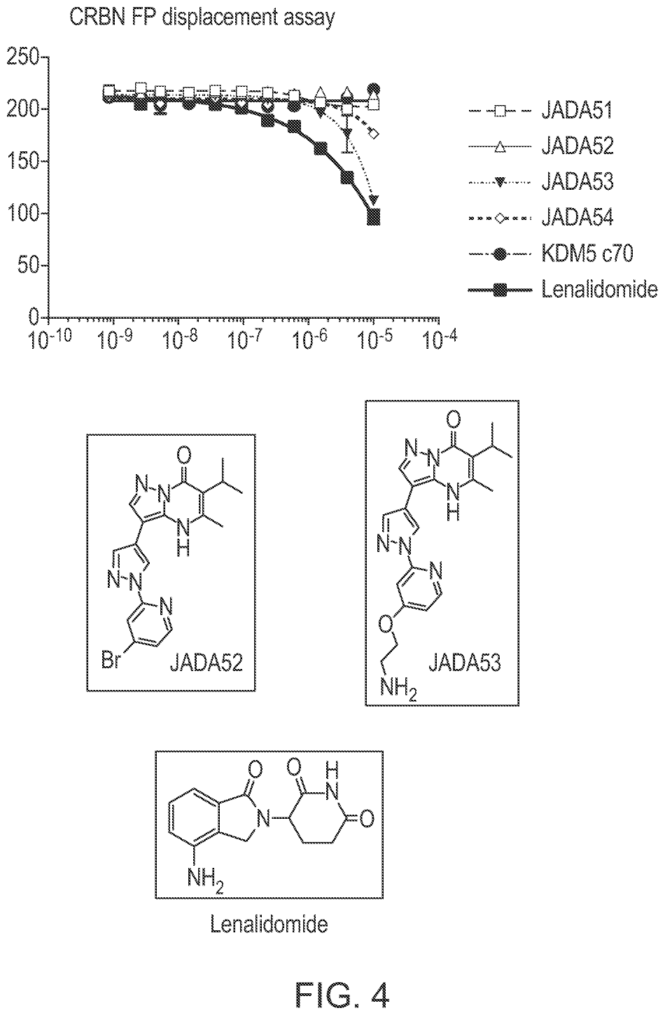

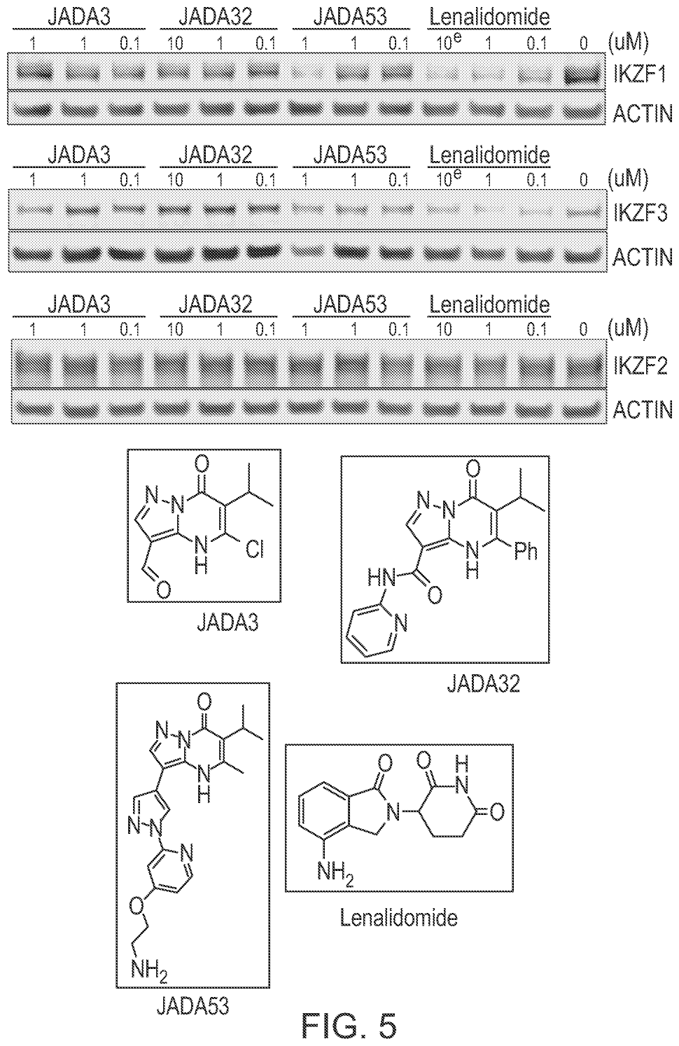

As shown in FIG. 4, exemplary compound JADA-53 binds CRBN. Exemplary close analogues had low activity against CRBN (FIG. 4). For the IKZF Western Blot, the cell is Jurkat, the exemplary compounds for treatment are JADA3, 32, 53, and lenalidomide, and the time point is 24 hours (FIG. 5). Exemplary compound JADA53 also degraded IKZF1/3 in cells, which indicates that the compound interacts with CRBN in cell.

Conditions for CRBN Fluorescence Polarization (FP) displacement assay: Atto565-conjugated lenalidomide (10 nM) was mixed with increasing concentration of purified his6-DDB1AB-his6—CRBN (10 μM final top concentration, 2-fold, 23-point dilution and DMSO control) in 384-well microplates (Corning, 4514) and incubated for 15 minutes (min) at room temperature (rt). The change in fluorescence polarization was monitored using a PHERAstar® FS microplate reader (BMG Labtech) for 20 min in 120 s cycles. The Atto565-lenalidomide bound fraction was calculated and the K_{d} was obtained from a fit in GraphPad Prism 7. Compounds in Atto565-Lenalidomide displacement assay were dispensed in a 384-well microplate (Corning®, 4514) using D300e Digital Dispenser (HP) normalized to 2% DMSO into 10 nM Atto565-Leanlidomide, 100 nM DDB1ΔB-CRBN, 50 mM Tris pH 7.5, 200 mM NaCl, 0.1% Pluronic® F-68 solution (Sigma), 0.5 mg/ml BSA (Sigma) containing 2% DMSO (4% DMSO final). The change in fluorescence polarization was monitored using a PHERAstar® FS microplate reader (BMG Labtech) for 20 min in 200 s cycles. IC₅₀ values estimated using variable slope equation in GraphPad Prism 7.

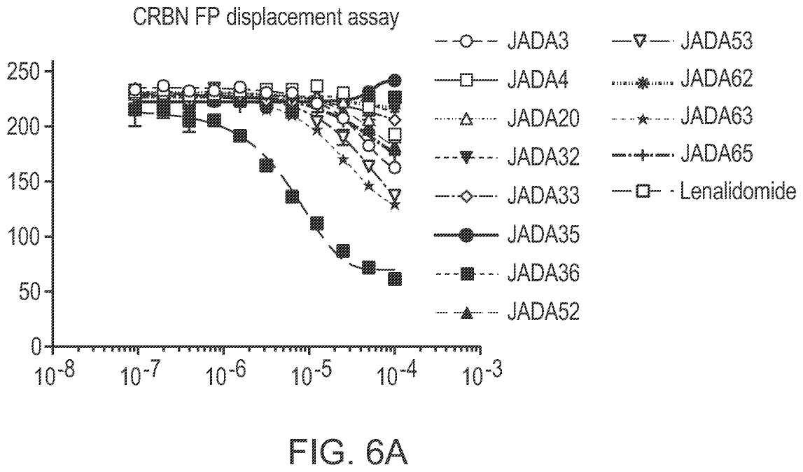

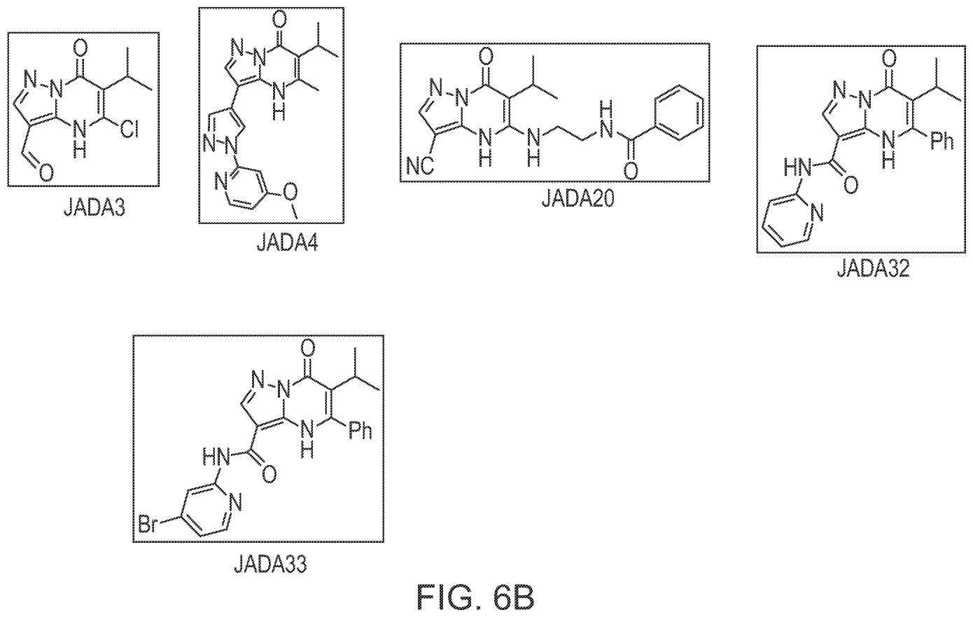

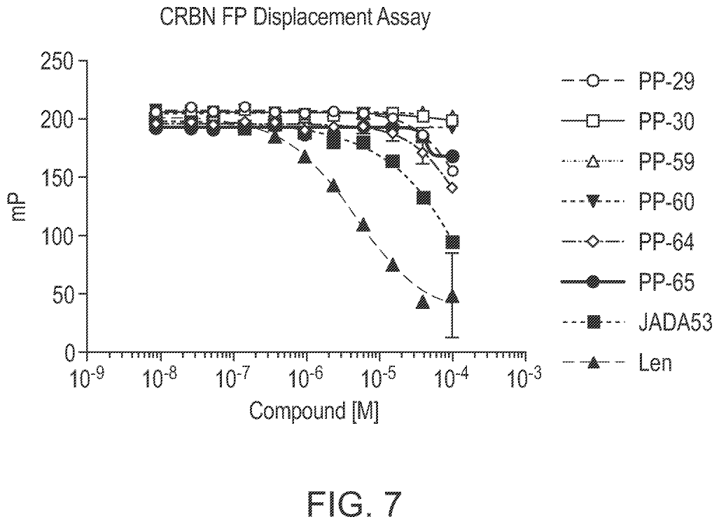

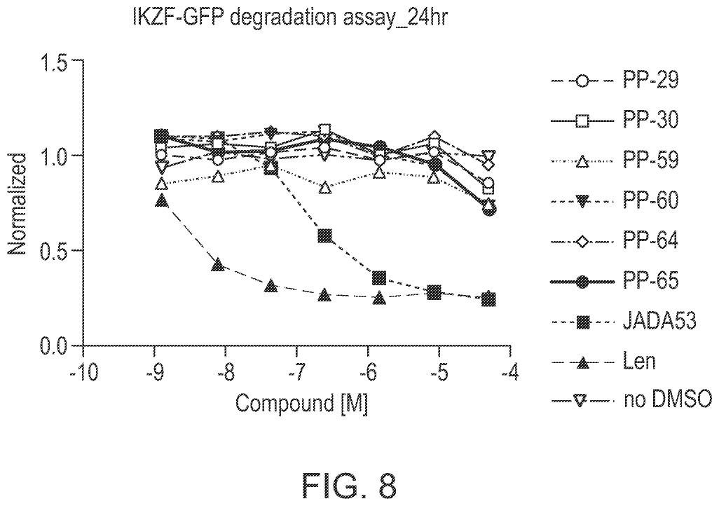

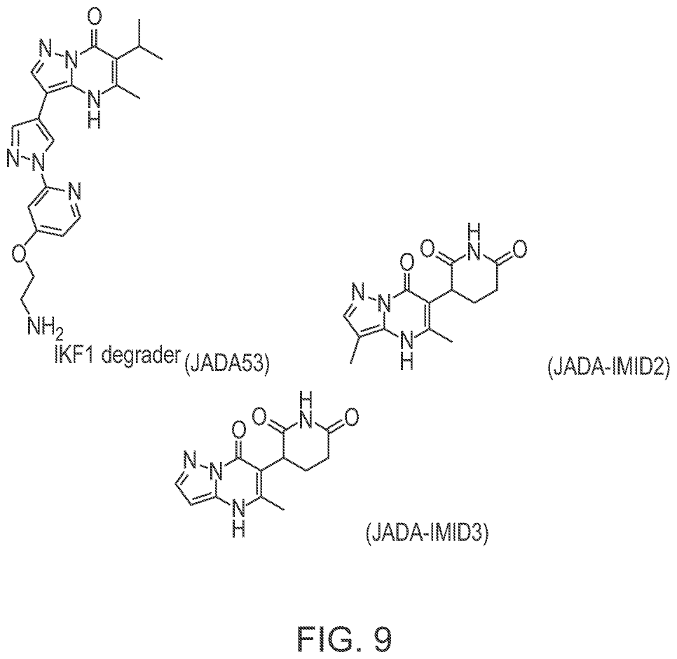

Results of a CRBN Fluorescence Polarization (FP) displacement assay of tested exemplary compounds JADA3, JADA4, JADA20, JADA32, JADA33, JADA35, JADA36, JADA52, JADA53, JADA62, JADA63, JADA65, and positive control lenalidomide at different concentrations [M], measured in [M] per Mp, are shown in FIGS. 6A and 6B. Other exemplary compound analogues have been tested, and some of them binded to CRBN. The results of a CRBN Fluorescence Polarization (FP) displacement assay of tested exemplary compounds PP-29, PP-30, PP-59, PP-60, PP-64, PP-65, JADA53, and positive control lenalidomide are shown in FIG. 7. Exemplary compound analogues with only the bottom part of JADA53 also binded CRBN. The results of a 24-hour IKZF-GFP (green fluorescent protein) degradation assay upon treatment with tested exemplary compounds PP-29, PP-30, PP-59, PP-60, PP-64, PP-65, JADA53, positive control lenalidomide, and control without DMSO at different concentrations [M] are shown in FIG. 8. FIG. 9 shows the chemical structures of exemplary compounds IZKF1 degrader, JADA-IMID2, and JADA-IMID3.

Conditions for CRBN Fluorescence Polarization (FP) displacement assay: Assays were performed with minimal modifications from the manufacturer's protocol (PerkinElmer®, USA). All reagents were diluted in 50 mM HEPES, 150 mM NaCl, 0.1% w/v BSA, 0.01% w/v Tween®20, pH 7.5 and allowed to equilibrate to room temperature prior to addition to plates. After addition of Alpha beads to master solutions all subsequent steps were performed under low light conditions. A 2× solution of components with final concentrations of His-CRBN at 50 nM, Ni-coated Acceptor Bead at 10 μg/ml, and 15 nM biotinylated-Pomalidomide was added in 10 μL to 384-well plates (AlphaPlate-384, PerkinElmer®, USA). Plates were spun down at 150×g, 100 nL of compound in DMSO from stock plates were added by pin transfer using a Janus® Workstation (PerkinElmer®, USA). The streptavidin-coated donor beads (10 μg/ml final) were added as with previous the solution in a 2×, 10 μL volume. Following this addition, plates were sealed with foil to prevent light exposure and evaporation. The plates were spun down again at 150 g. Plates were incubated at room temperature (rt) for 1 hour and then read on an Envision 2104 (PerkinElmer®, USA) using the manufacturer's protocol.

Conditions for Western blot:

Jurkat cells were seeded in 6 well plates, followed by the compound treatment for 24 hours. Primary and secondary antibodies used included anti-IKZF1 at 1:1000 dilution, anti-IKZF2 at 1:1,000 dilution, anti-IKZF3 at 1:1000 dilution, anti-ACTIN at 1:5,000 dilution, IRDye®680 Donkey anti-mouse at 1:5,000 dilution (926-68072, LiCor®, Lot: C61116-05) and IRDye®800 Goat anti-rabbit at 1:5,000 dilution (926-32211, LiCor®, Lot: C70301-05).

Conditions for IKZF1 Cellular Degradation Assay:

IKZF1Δ were subcloned into mammalian pcDNA5/FRT Vector (Ampicillin and Hygromycin B resistant) modified to contain MCS-eGFP-P2A-mCherry. Stable cell lines expressing eGFP-protein fusion and mCherry reporter were generated using Flip-In™ 293 system. Plasmid (0.3 μg) and pOG44 (4.7 μg) DNA were preincubated in 100 μL of Opti-MEM™I (Gibco®, Life Technologies™) media containing 0.05 mg/ml Lipofectamine 2000 (Invitrogen™) for 20 min and added to Flip-In™ 293 cells containing 1.9 ml of Dulbecco's Modified Eagle Medium (DMEM) media (Gibco®, Life Technologies™) per well in a 6-well plate format (Falcon®, 353046). Cells were propagated after 48 hours and transferred into a 10 cm² plate (Corning®, 430165) in DMEM media containing 50 μg/ml of Hygromycin B (REF 10687010, Invitrogen™) as a selection marker. Following 2-3 passage cycle FACS (FACSAria™ II, BD) was used to enrich for cells expressing eGFP and mCherry.

Cells were seeded at 30-50% confluency in either 24, 48 or 96 well plates (3524, 3548, 3596 respectively, Costar®) a day before compound treatment. Titrated compounds (see, Figure legends) were incubated with cells for 5 h following trypsinization and resuspension in DMEM media, transferred into 96-well plates (353910, Falcon®) and analyzed by flow cytometer (guava easyCyte™ HT, Millipore™). Signal from minimal 3000 events per well was acquired and the eGFP and mCherry florescence monitored. Data was analyzed using FlowJo® (FlowJo®, LCC). Forward and side scatter outliers, frequently associated with cell debris, were removed leaving >90% of total cells, followed by removal of eGFP and mCherry signal outliers, leaving 88-90% of total cells creating the set used for quantification. The eGFP protein abundance relative to mCherry was then quantified as a ten-fold amplified ratio for each individual cell using the formula: 10×eGFP/mCherry. The median of the ratio was then calculated per set, normalized to the median of the DMSO ratio.

Conditions for TMT LC-MS3 Mass Spectrometry Assay:

MM. 1s cell were treated with DMSO and 10 μM JADA53 in cell culture for 12 hours and 24 hours. Cells were harvested by centrifugation. Lysis buffer (8 M Urea, 1% SDS, 50 mM Tris pH 8.5, Protease and Phosphatase inhibitors from Roche®) was added to the cell pellets to achieve a cell lysate with a protein concentration between 2-8 mg mL⁻¹. A micro-BCA assay (Pierce™) was used to determine the final protein concentration in the cell lysate. 200 μg proteins for each sample were reduced and alkylated as previously described. Proteins were precipitated using methanol/chloroform. In brief, four volumes of methanol were added to the cell lysate, followed by one volume of chloroform, and finally three volumes of water. The mixture was vortexed and centrifuged to separate the chloroform phase from the aqueous phase. The precipitated protein was washed with one volume of ice-cold methanol. The washed precipitated protein was allowed to air dry. Precipitated protein was resuspended in 4 M Urea, 50 mM Tris pH 8.5. Proteins were first digested with LysC (1:50; enzyme:protein) for 12 hours at 25° C. The LysC digestion was diluted down in 1 M Urea, 50 mM Tris pH 8.5 and then digested with trypsin (1:100; enzyme:protein) for another 8 hours at 25° C. Peptides were desalted using a C₁₈ solid phase extraction cartridges (Waters). Dried peptides were resuspended in 200 mM 3-[4-(2-hydroxyethyl)-1-piperazinyl]propanesulfonic acid (EPPS), pH 8.0. Peptide quantification was performed using the micro-BCA assay (Pierce™). The same amount of peptide from each condition was labelled with tandem mass tag (TMT) reagent (1:4; peptide:TMT label) (Pierce™). The 10-plex labelling reactions were performed for 2 hours at 25° C. Modification of tyrosine residue with TMT was reversed by the addition of 5% hydroxyl amine for 15 min at 25° C. The reaction was quenched with 0.5% TFA and samples were combined at a 1:1:1:1:1:1:1:1:1:1 ratio. Combined samples were desalted and offline fractionated into 96 fractions using an aeris peptide xb-c18 column (phenomenex®) at pH 8.0. Fractions were recombined in a non-continuous manner into 24 fractions and every second fraction was used for subsequent mass spectrometry analysis.

Data were collected using an Orbitrap Fusion™ Lumos™ mass spectrometer (Thermo Fisher Scientific, San Jose, Calif., USA) coupled with a Proxeon EASY-nLC™ 1200 LC pump (Thermo Fisher Scientific). Peptides were separated on a 75 μm inner diameter microcapillary column packed with 35 cm of Accucore™ C18 resin (2.6 μm, 100 Å, ThermoFisher Scientific). Peptides were separated using a 3-hour gradient of 6-27% acetonitrile in 0.125% formic acid with a flow rate of 400 nL/min.

Each analysis used an MS³-based TMT method as described previously in McAlister et al., Anal. Chem. 2014, 86, 7150-7158. The data were acquired using a mass range of m/z 350-1350, resolution 120,000, AGC target 1×10⁶, maximum injection time 100 ms, dynamic exclusion of 120 seconds for the peptide measurements in the Orbitrap. Data dependent MS²spectra were acquired in the ion trap with a normalized collision energy (NCE) set at 35%, AGC target set to 1.8×10⁴ and a maximum injection time of 120 ms. MS³ scans were acquired in the Orbitrap with a HCD collision energy set to 55%, AGC target set to 1.5×10⁵, maximum injection time of 150 ms, resolution at 50,000 and with a maximum synchronous precursor selection (SPS) precursors set to 10.

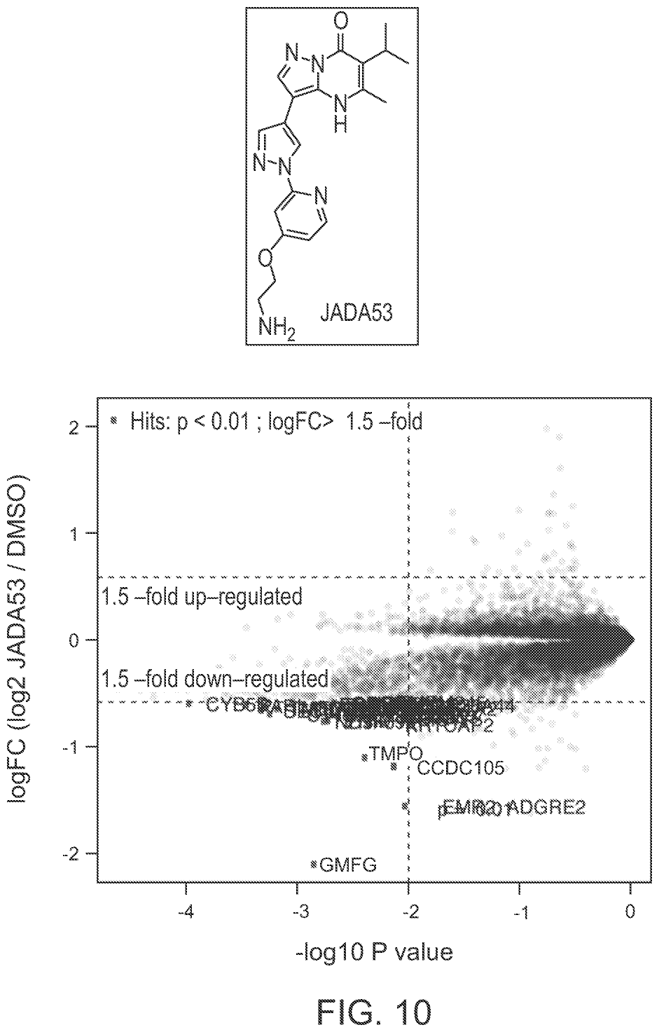

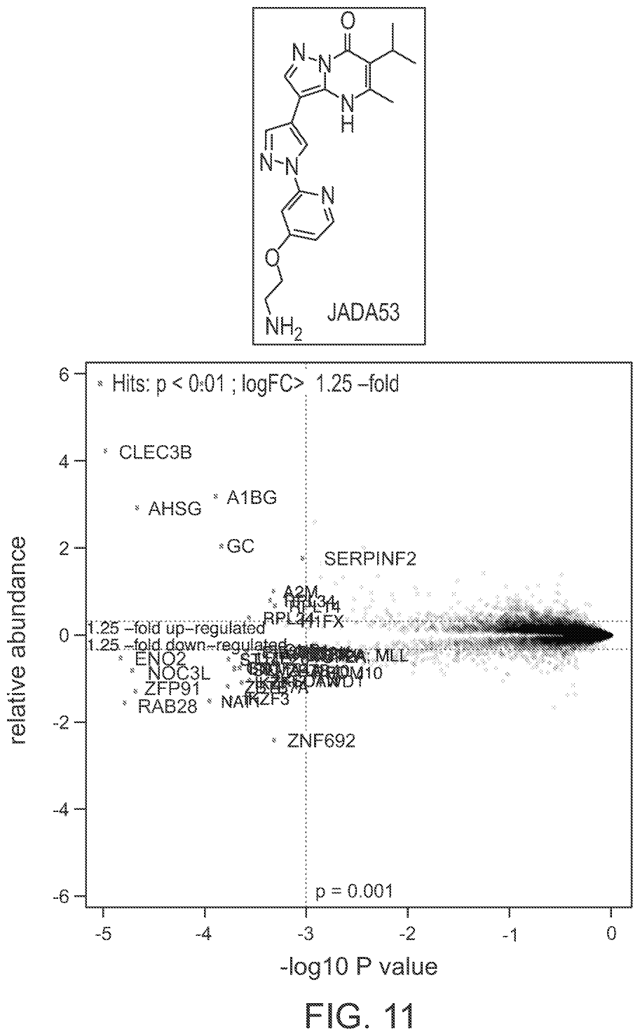

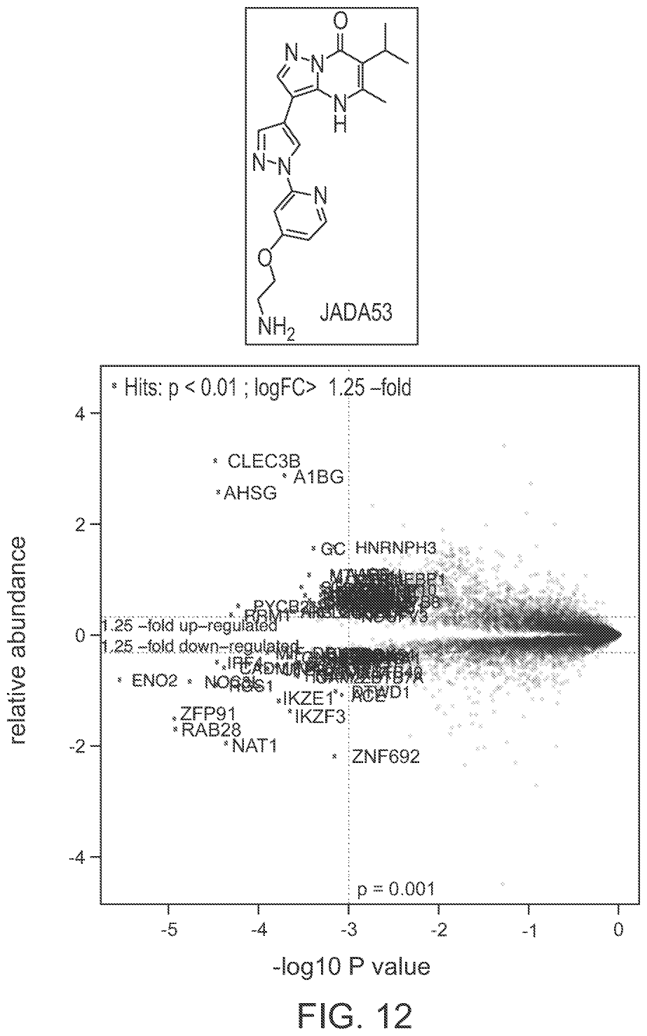

Results of the TMT LC-MS3 mass spectrometry experiments showing fold change in relative abundance comparing treatment with exemplary compound JADA53 to DMSO control treatment for Kelly cells are provided (FIGS. 10 and 11) and provided for MM.1 human multiple myeloma (MM) cell lines (FIG. 12).

Conditions for time-resolved fluorescence resonance energy transfer (TR-FRET) assay:

Compounds were dispensed in a 384-well microplate (Corning, 4514) using D300e Digital Dispenser (HP) normalized to 2% DMSO into 200 nM biotinylated pomalidomide,100 nM His6-DDB1ΔB-His₆-CRBN_{BODIPY}catcher and 2 nM terbium-coupled streptavidin (Invitrogen™) in a buffer containing 50 mM Tris pH 7.5, 200 mM NaCl, 0.1% Pluronic® F-68 solution (Sigma) and 2% DMSO (4% DMSO final). Before TR-FRET measurements were conducted, the reactions were incubated for 15 min at rt. After excitation of terbium fluorescence at 337 nm, emission at 490 nm (terbium) and 520 nm (boron-dipyrromethene (BODIPY)) were recorded with a 70 μs delay over 600 μs to reduce background fluorescence and the reaction was followed over 30 200-second cycles of each data point using a PHERAstar™ FS microplate reader (BMG Labtech). The TR-FRET signal of each data point was extracted by calculating the 520/490 nm ratio.

Compounds of Formulae (I) or (II) may be prepared using the synthetic schemes and procedures described in detail below.

### Example 2

### Synthetic Methods

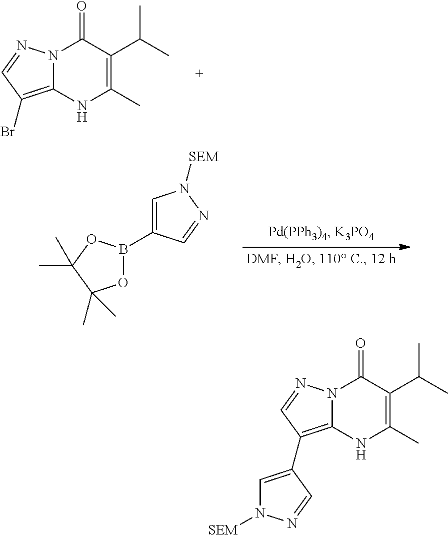 [SMILES: Cc1[nH]c2c(Br)cnn2c(=O)c1C(C)C ; CC1(C)OB(c2cnn(COCC[Si](C)(C)C)c2)OC1(C)C ; Cc1[nH]c2c(-c3cnn(COCC[Si](C)(C)C)c3)cnn2c(=O)c1C(C)C]

A mixture of 3-bromo-6-isopropyl-5-methylpyrazolo[1,5-a]pyrimidin-7(4H)-one (540 mg, 2 mmol), 4-(4,4,5,5-tetramethyl-1,3,2-dioxaborolan-2-yl)-1-((2-(trimethylsilypethoxy)methyl)-1H-pyrazole (972 mg, 3 mmol), Pd(PPh₃)₄ (230 mg, 0.2 mmol) and K₃PO₄ (1.76 g, 8 mmol) in DMF/H20 (10 ml/1 ml) was heated at 110° C. for 12 hours under N₂ atmosphere. Then the mixture was extracted with ethyl acetate (EA) (50 ml×3), and the combined organic layers were dried over Na₂SO₄ and evaporated under reduced pressure. The resulting residue was purified by ISCO (pure EA) to get the product 70 mg (10% yield). LC MS (ESI) m/z: 388.3 [M+H⁺].

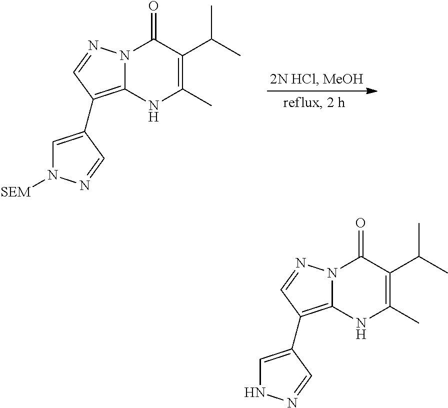 [SMILES: Cc1[nH]c2c(-c3cnn(COCC[Si](C)(C)C)c3)cnn2c(=O)c1C(C)C ; Cc1[nH]c2c(-c3cn[nH]c3)cnn2c(=O)c1C(C)C]

A mixture of 6-isopropyl-5-methyl-3-(1-((2-(trimethylsilyl)ethoxy)methyl)-1H-pyrazol-4-yl)pyrazolo[1,5-a]pyrimidin-7(4H)-one (70 mg, 0.18 mmol) in MeOH (2 ml) and 2N HCl (0.2 ml) was heated at reflux for 2 hours. Then saturated aqueous NaHCO₃ was added to adjust the pH value 7, and the mixture was extracted with EA (50 ml×3). The combined organic layers were dried over Na₂SO₄ and evaporated under reduced pressure. The resulting residue was purified by ISCO (DCM/MeOH=10/1) to get the product (40 mg, 83% yield) as a yellow solid. LC MS (ESI) m/z: 258.3 [M+H⁺].

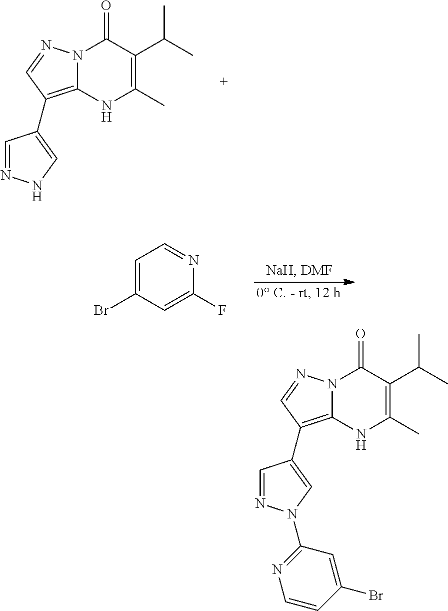 [SMILES: Cc1[nH]c2c(-c3cn[nH]c3)cnn2c(=O)c1C(C)C ; Fc1cc(Br)ccn1 ; Cc1[nH]c2c(-c3cnn(-c4cc(Br)ccn4)c3)cnn2c(=O)c1C(C)C]

To a solution of 6-isopropyl-5-methyl-3-(1H-pyrazol-4-yl)pyrazolo[1,5-a]pyrimidin-7(4H)-one (40 mg, 0.15 mmol) in DMF (2 ml), NaH (60%, 6.8 mg, 0.17 mmol) was added at 0° C., and the mixture was stirred at rt for 30 min. 4-Bromo-2-fluoropyridine (26.7 mg, 0.15 mmol) was added to the mixture at 0° C., and the reaction was stirred at 35° C. overnight. The reaction mixture was poured into H₂O (10 ml), extracted with EA (50 ml×3), dried over Na₂SO₄ and evaporated under reduced pressure. The resulting residue was purified by ISCO (DCM/MeOH=10/1) to get 3-(1-(4-bromopyridin-2-yl)-1H-pyrazol-4-yl)-6-isopropyl-5-methylpyrazolo[1,5-a]pyrimidin-7(4H)-one as a white solid 25 mg (48% yield). LC MS (ESI) m/z: 413.0 [M+H⁺].

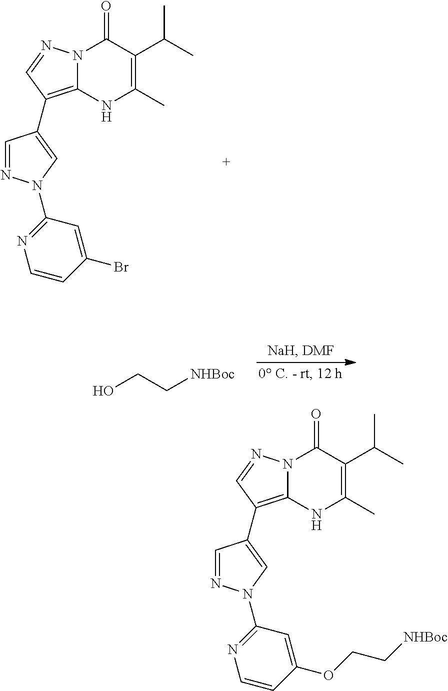 [SMILES: Cc1[nH]c2c(-c3cnn(-c4cc(Br)ccn4)c3)cnn2c(=O)c1C(C)C ; CC(C)(C)OC(=O)NCCO ; Cc1[nH]c2c(-c3cnn(-c4cc(OCCNC(=O)OC(C)(C)C)ccn4)c3)cnn2c(=O)c1C(C)C]

To a solution of tert-butyl (2-hydroxyethyl)carbamate (10 mg, 0.06 mmol) in DMF (1 ml), NaH (3 mg, 0.07 mmol) was added at 0° C., and the mixture was stirred at rt for 30 min. 3-(1 -(4-Bromopyridin-2-yl)-1H-pyrazol -4-yl)-6-isopropyl-5 -methylpyrazolo [1,5 -a]pyrimidin-7(4H)-one (25 mg, 0.06 mmol) was added to the mixture at 0° C., and the reaction was kept stirring at 35° C. overnight. The reaction mixture was poured into H₂O (10 mL), and the mixture was extracted with EA (50 ml×3), dried over Na₂SO₄ and evaporated under reduced pressure. The resulting residue was purified by ISCO (DCM/MeOH=10/1) to get the product as a white solid 15 mg (43% yield). LC MS (ESI) m/z: 494.3 [M+H⁺].

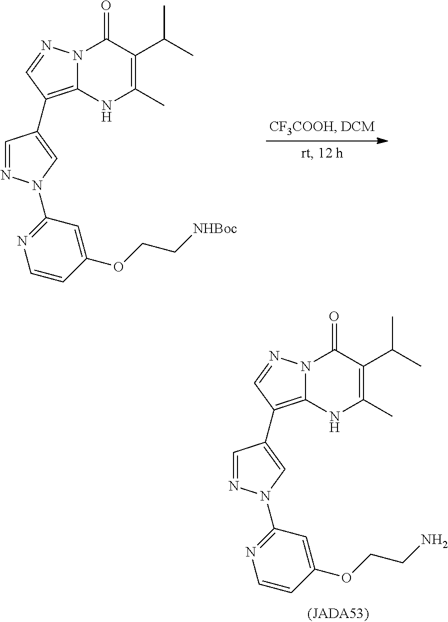 [SMILES: Cc1[nH]c2c(-c3cnn(-c4cc(OCCNC(=O)OC(C)(C)C)ccn4)c3)cnn2c(=O)c1C(C)C ; Cc1[nH]c2c(-c3cnn(-c4cc(OCCN)ccn4)c3)cnn2c(=O)c1C(C)C]

To a solution of tert-butyl (2-((2-(4-(6-isopropyl-5-methyl-7-oxo-4,7-dihydropyrazolo[1,5-a]pyrimidin-3-yl)-1H-pyrazol-1-yl)pyridin-4-yl)oxy)ethyl)carbamate (15 mg, 0.03 mmol) in DCM (1 ml), CF₃COOH (0.25 ml)was added and the reaction was stirred overnight. The solvent was removed under reduced pressure, and the crude product was purified by HPLC to get JADA53 as a white solid 7 mg (75% yield). LC MS (ESI) m/z: 394.5 [M+H⁺].

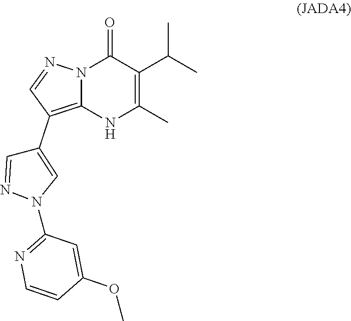 [SMILES: COc1ccnc(-n2cc(-c3cnn4c(=O)c(C(C)C)c(C)[nH]c34)cn2)c1]

Compound JADA4 was was prepared in an analogous manner to above exemplary compound JADA53 from 3-(1-(4-bromopyridin-2-yl)-1H-pyrazol-4-yl)-6-isopropyl-5-methylpyrazolo[1,5-a]pyrimidin-7(4H)-one.

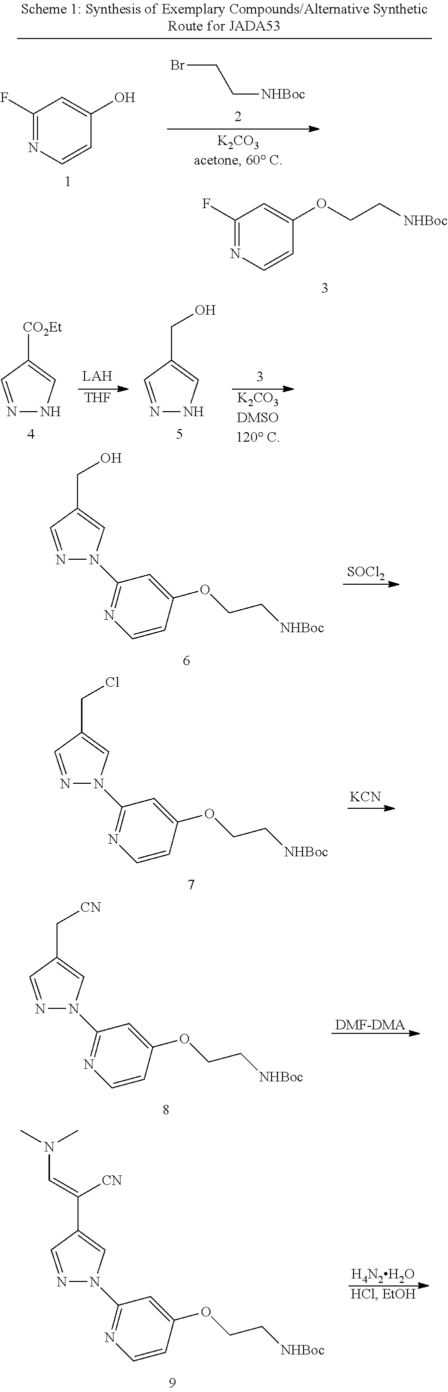

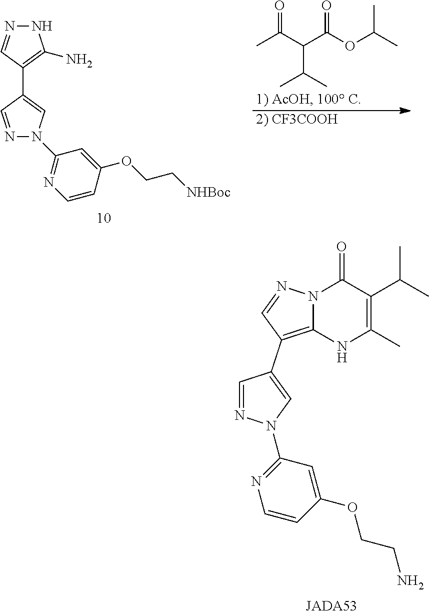

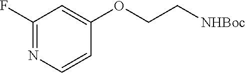 [SMILES: CC(C)(C)OC(=O)NCCOc1ccnc(F)c1]

### tert-Butyl (2-((2-fluoropyridin-4-yl)oxy)ethyl)carbamate (3)

To a solution of 2-fluoropyridin-4-ol (1) (1.13 g, 10 mmol) in acetone (30 mL) was added K₂CO₃ (2.76 g, 20 mmol) and compound 2 (2.46 g, 11 mol). The reaction mixture was heated under reflux for 16 hours. After cooling, the mixture was diluted with EA and filtered through Celite®. The solvent was removed under reduded pressure to give compound 3 (2.5 g, 99%).

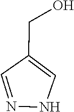

### (1H-pyrazol-4-yl)methanol (5)

To an oven-dried flask containing 4-ethylpyrazole carboxylate (4) (10 g, 71.40 mmol) was added anhydrous THF (100 ml) under argon atmosphere. A solution of lithium aluminium hydride (LAH) in THF (1 M, 100 ml, 100 mmol) was then added dropwise to the mixture. The resulting mixture was stirred overnight at rt. The reaction mixture was cooled in an ice-bath and quenched with water (3.8 ml), followed by 15% sodium hydroxide (3.8 ml) and water (11.4 ml). The resulting mixture was dried over Na₂SO₄, filtered, and concentrated under reduced pressure to give the title compound (5 g, 71%).

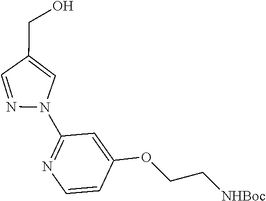 [SMILES: CC(C)(C)OC(=O)NCCOc1ccnc(-n2cc(CO)cn2)c1]

### tert-Butyl(2-((2-(4-(hydroxymethyl)-1H-pyrazol-1-yl)pyridin-4-yl)oxy)ethyl) carbamate (6)

To a solution of compound 5 (1 g, 10.2 mmol) in anhydrous DMSO (20 mL) was added K₂CO₃ (2.82 g, 20.4 mmol) and comound 3 (2.87 g, 11.2 mmol). The reaction mixture was stirred at 120° C. for 3 days. The mixture was diluted with EA and washed with water and brine. The organic layer was dried over Na₂SO₄, filtered, and concentrated under reduced pressure. The crude product was purified by SiO₂ column chromatography (EA/MeOH=4/1) to give compound 6 as a white solid (1 g, 33%).

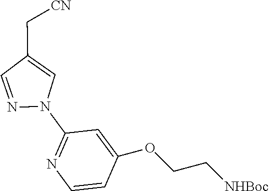 [SMILES: CC(C)(C)OC(=O)NCCOc1ccnc(-n2cc(CC#N)cn2)c1]

tert-Butyl (2-((2-(4-(chloromethyl)-1H-pyrazol-1-yl)pyridin-4-yl)oxy)ethyl) carbamate (7)

To a solution of compound 6 (0.33 g, 1 mmol) in DCM (10 mL) was added SOCl₂ (357 mg, 3 mmol) dropwise at 0° C. The reaction mixture was stirred for 4 hours at 0° C. and then concentrated under reduced pressure. The residue was diluted with EA (30 mL) and washed with aqueous K₂CO₃ (1 M). The organic layer was separated, and the aqueous layer was extracted with EA. The combined organic layers were dried over Na₂SO₄, filtered, and concentrated under reduced pressure to give crude compound 7.

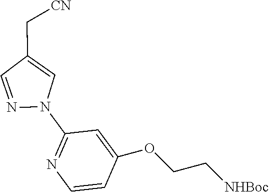 [SMILES: CC(C)(C)OC(=O)NCCOc1ccnc(-n2cc(CC#N)cn2)c1]

### tert-Butyl (2-((2-(4-(cyanomethyl)-1H-pyrazol-1-yl)pyridin-4-yl)oxy)ethyl) carbamate (8)

KCN (163 mg, 2.5 mmol) was added in portions to a stirred solution of compound 7 g, 0.85 mmol) in DMSO/H₂O (3 mL/1 mL). The resulting mixture was heated at 50° C. for 5 hours. The reaction mixture was cooled and diluted with EA (50 mL). The organic layer was separated, and the aqueous layer was ex with EA (30 mL×3). The combined organic layers were dried dried over Na₂SO₄, filtered, and concentrated under reduced pressure. The crude product was purified by preparative (prep)-HPLC to give title compound (90 mg, 30%) as a white solid.

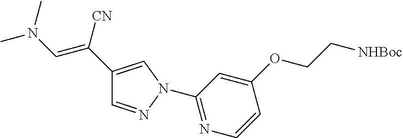 [SMILES: CN(C)/C=C(\C#N)c1cnn(-c2cc(OCCNC(=O)OC(C)(C)C)ccn2)c1]

### tert-Butyl (Z)-(2-((2-(4-(1-cyano-2-(dimethylamino)vinyl)-1H-pyrazol-1-yl)pyridin-4-yl)oxy)ethyl)carbamate (9)

A mixture of compound 8 (90 mg, 0.26 mmol) in DMF-DMA (2.0 mL) was heated at 140° C. for 30 minutes under microwave irradiation. The mixture was concentrated under reduced pressure to afford crude compound 9, which was used in the next step without further purification.

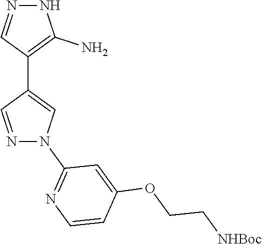 [SMILES: CC(C)(C)OC(=O)NCCOc1ccnc(-n2cc(-c3cn[nH]c3N)cn2)c1]

### tert-Butyl (2-((2-(5′-amino-1H,1′H-[4,4′-bipyrazol]-1-yl)pyridin-4-yl)oxy)ethyl)carbamate (10)

To a stirred solution of hydrazine monohydrate (1 mL) in EtOH (10 mL) was slowly added concentrated HCl (12 M) until pH 3, followed by compound 9 (0.6 g, 1.5 mmol). The resultant yellow solution was heated at 70° C. for 1 hour. The reaction was allowed to cool to rt, and aqueous K₂CO₃ (1 M) was added until pH 9. The resulting mixture was then extracted with EA (20 mL×3). The combined organic layers were dried over Na₂SO₄, filtered, and concentrated under reduced pressure. The residue was purified by prep-HPLC to give compound 10 (0.5 g, 85% yield) as a white solid.

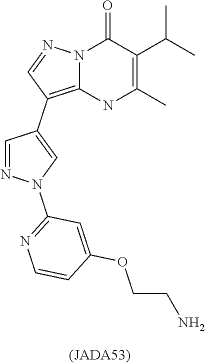 [SMILES: Cc1[nH]c2c(-c3cnn(-c4cc(OCCN)ccn4)c3)cnn2c(=O)c1C(C)C]

Compound 10 (300 mg, 0.78 mmol) and isopropyl 2-acetyl-3-methylbutanoate (400 mg, 3 eq) were dissolved in AcOH (5 mL), and the mixture was stirred at 100° C. overnight. The reaction was allowed to cool to rt, and the crude mixture was concentrated under reduced pressure. Both the Boc protected JADA53 and JADA53 were formed during the reaction. The resulting residue was dissolved in TFA/DCM (1:1), and the mixture was stirred at rt for 1 hour. The solvent was under reduced pressure, and the crude residue was purified by prep-HPLC (trace TFA) to give compound JADA53 (20 mg, 86% yield) as a white solid.

Certain steps of the procedure provided in Scheme 1 (above) can be applied to the synthesis of JADA4. For example, the BOC group on each of the above intermediates were removed, and the obtained compounds were tested. The intermediates may also be alkylated and derivatized further as well.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula: 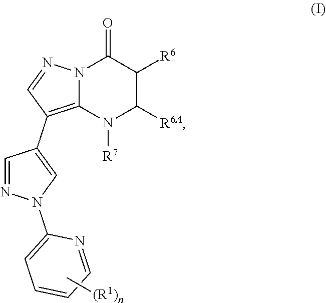
or a pharmaceutically acceptable salt, co-crystal, tautomer, stereoisomer, solvate, hydrate, polymorph, isotopically enriched derivative, or prodrug thereof, wherein each instance of le is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, or O(CH₂)ₓR^{1A};
provided at least one instance of R¹ is —O(CH₂)ₓR^{1A};
R⁶ is unsubstituted isopropyl, —(CH₂)C(═O)OMe, or —(CH₂)₂OH;
R^{6A} is optionally substituted alkyl;
R⁷ is hydrogen, optionally substituted acyl, optionally substituted alkyl, or a nitrogen protecting group;
R^{1A} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A}, —N(R^{B})₂, or —SR^{A}, as valency permits;
each instance of R^{A} is independently hydrogen, optionally substituted acyl, optionally substituted alkyl, an oxygen protecting group when attached to an oxygen atom, or a sulfur protecting group when attached to a sulfur atom;
each instance of R^{B} is independently hydrogen, optionally substituted acyl, optionally substituted alkyl, or a nitrogen protecting group;
n is 1, 2, 3, or 4; and
x is 0, 1, 2, 3, 4, 5, or 6, provided that when R⁶ is isopropyl, R^{6A} is methyl, R⁷ is hydrogen, and n is 1, R¹ is not methyl or hydroxymethyl.

2. The compound of claim 1, wherein the compound is: 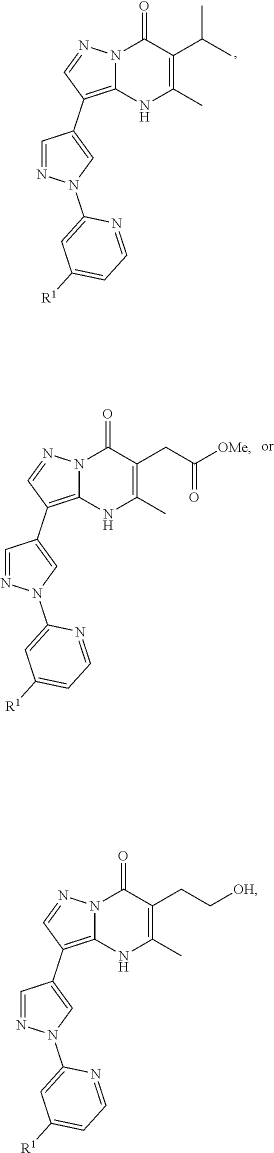
or a pharmaceutically acceptable salt, co-crystal, tautomer, stereoisomer, solvate, hydrate, polymorph, isotopically enriched derivative, or prodrug thereof.

3. The compound of claim 1, wherein:
R¹ is —O(CH₂)₂N(R^{B})₂; or
at least one instance of R^{B} is hydrogen; or
both instances of R^{B} are unsubstituted C₁₋₆ alkyl; or
R¹ is —OMe or —O(CH₂)₂NH₂; or
n is 1 or 2; or
R6 is unsubstituted isopropyl; or
R^{6A} is unsubstituted methyl; or
R⁷ is hydrogen.

4.-13. (canceled)

14. The compound of claim 1, wherein the compound is: 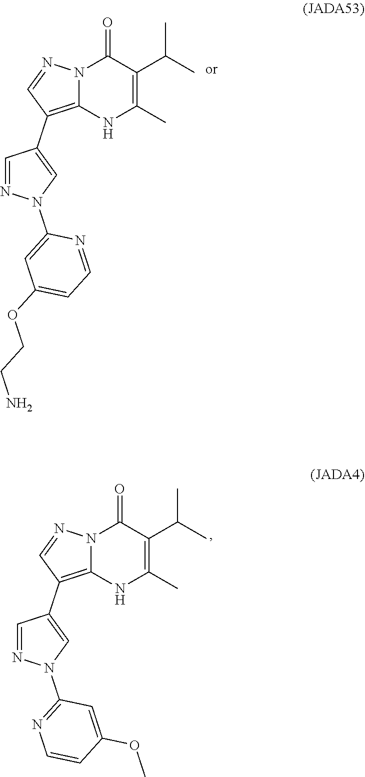 [SMILES: Cc1[nH]c2c(-c3cnn(-c4cc(OCCN)ccn4)c3)cnn2c(=O)c1C(C)C ; COc1ccnc(-n2cc(-c3cnn4c(=O)c(C(C)C)c(C)[nH]c34)cn2)c1]
or a pharmaceutically acceptable salt, co-crystal, tautomer, stereoisomer, solvate, hydrate, polymorph, isotopically enriched derivative, or prodrug thereof.

15. A compound of Formula (II): 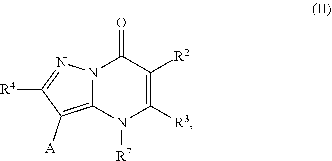
or a pharmaceutically acceptable salt, co-crystal, tautomer, stereoisomer, solvate, hydrate, polymorph, isotopically enriched derivative, or prodrug thereof, wherein:
A is hydrogen, optionally substituted alkyl, —C(═O)N(R⁵)₂, —C(═O)(R⁵), —C(═O)OR⁵, or —CN;
R² is hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{2B}, —N(R^{2A})₂, or —SR^{2B};
R³ is hydrogen, halogen, optionally substituted acyl, optionally substituted alkyl, optionally substituted alkenyl, optionally substituted alkynyl, optionally substituted carbocyclyl, optionally substituted aryl, optionally substituted heteroaryl, —OR^{2A}, —N(R^{2B})₂, or —SR^{2A};
R⁴ is hydrogen, halogen, optionally substituted acyl, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{2A}, —N(R^{2B})₂, —SR^{2A}, or —CN; and
each instance of R⁵ is independently hydrogen, optionally substituted alkyl, optionally substituted alkenyl, optionally substituted alkynyl, optionally substituted carbocyclyl, optionally substituted aryl, optionally substituted heterocyclyl, or optionally substituted heteroaryl;
R⁷ is hydrogen, optionally substituted acyl, optionally substituted alkyl, or a nitrogen protecting group;
each instance of R^{2A} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and
each instance of R^{2B} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group, or optionally two instances of R^{2A} are taken together with their intervening atoms to form a substituted or unsubstituted heterocyclic or substituted or unsubstituted heteroaryl ring.

16. The compound of claim 15, wherein R² is optionally substituted C₁₋₆ alkyl or optionally substituted heterocyclyl.

17. The compound of claim 16, wherein R² is unsubstituted isopropyl or

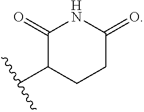 [SMILES: *C1CCC(=O)NC1=O]

18.-19. (canceled)

20. The compound of claim 15, wherein R³ is halogen, optionally substituted C₁₋₆ alkyl, optionally substituted phenyl, or —NH(optionally substituted C₁₋₆ alkyl).

21. The compound of claim 20, wherein R³ is —Cl, methyl, phenyl, or

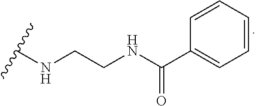 [SMILES: *NCCNC(=O)c1ccccc1]

22.-27. (canceled)

28. The compound of claim 15, wherein R⁴ is hydrogen.

29. The compound of claim 15, wherein A is hydrogen, optionally substituted C₁₋₆ alkyl, —C(═O)NH(R⁵), —C(═O)H, or —CN.

30. (canceled)

31. The compound of claim 29, wherein A is unsubstituted methyl.

32. (canceled)

33. The compound of claim 29, wherein A is —C(═O)NH(R⁵) and R⁵ is optionally substituted heteroaryl.

34. The compound of claim 33, wherein A is

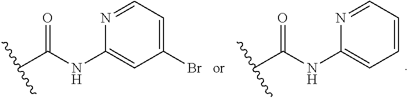 [SMILES: *C(=O)Nc1cc(Br)ccn1 ; *C(=O)Nc1ccccn1 ; "or" ; "."]

35.-36. (canceled)

37. The compound of claim 15, wherein the compound is: 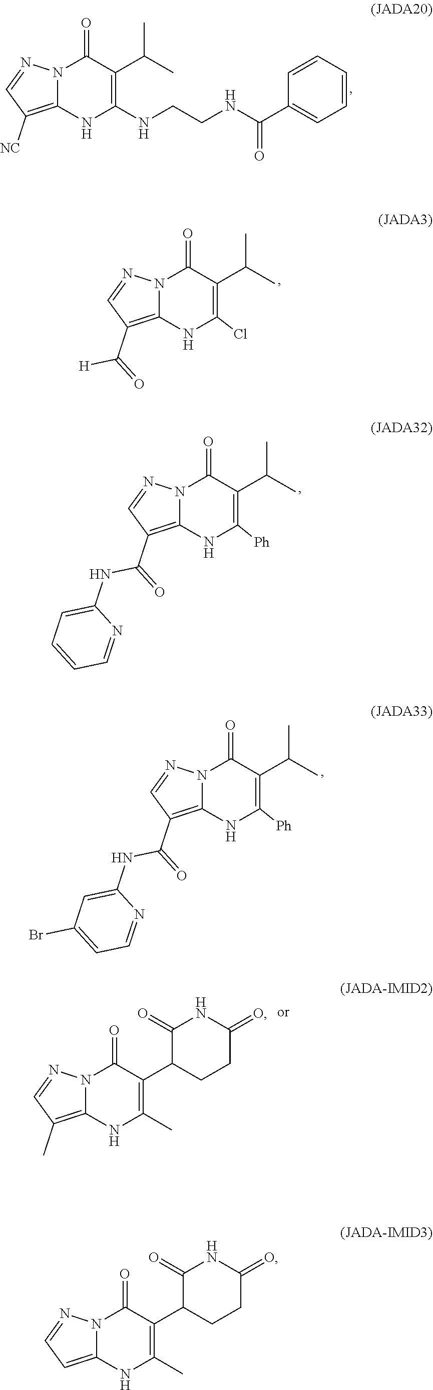
or a pharmaceutically acceptable salt, co-crystal, tautomer, stereoisomer, solvate, hydrate, polymorph, isotopically enriched derivative, or prodrug thereof.

38. A compound of any one of structures: 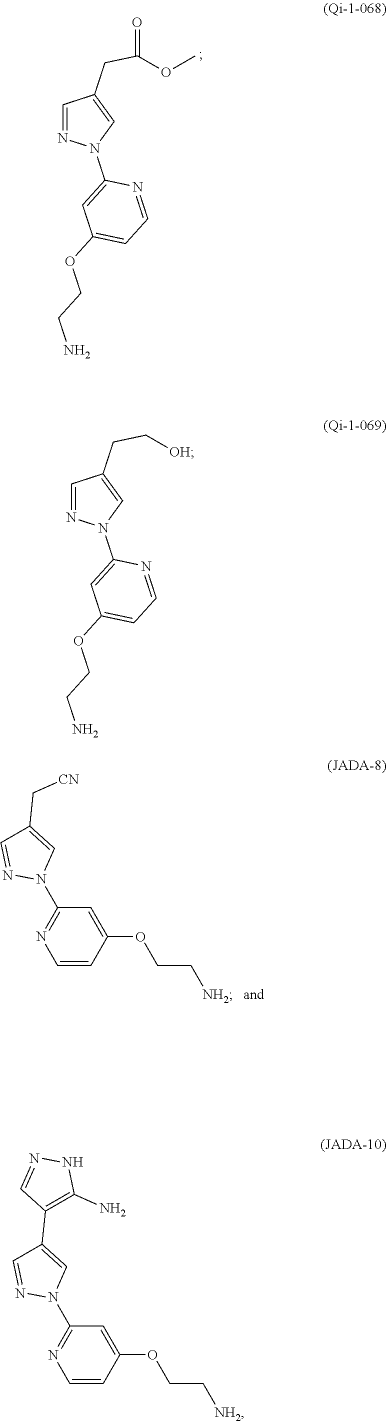 [SMILES: COC(=O)Cc1cnn(-c2cc(OCCN)ccn2)c1 ; NCCOc1ccnc(-n2cc(CCO)cn2)c1 ; N#CCc1cnn(-c2cc(OCCN)ccn2)c1 ; "and" ; NCCOc1ccnc(-n2cc(-c3cn[nH]c3N)cn2)c1]
or a pharmaceutically acceptable salt, co-crystal, tautomer, stereoisomer, solvate, hydrate, polymorph, isotopically enriched derivative, or prodrug thereof.

39. A pharmaceutical composition comprising a therapeutically effective amount of the compound of claim 1, 15, or 38, or a pharmaceutically acceptable salt, co-crystal, tautomer, stereoisomer, solvate, hydrate, polymorph, isotopically enriched derivative, or prodrug thereof, and optionally a pharmaceutically acceptable excipient.

40. (canceled)

41. A method of treating a proliferative disease in a subject in need thereof, the method comprising administering to the subject a therapeutically effective amount of the compound or pharmaceutically acceptable salt, co-crystal, tautomer, stereoisomer, solvate, hydrate, polymorph, isotopically enriched derivative, or prodrug or stereoisomer thereof of claim 1, 15, or 38.

42. The method of claim 41, wherein the proliferative disease is cancer.

43. The method of claim 42, wherein the cancer is a hematopoietic cancer or a solid tumor.

44. The method of claim 42, wherein the cancer is leukemia, multiple myeloma, myelodysplastic syndrome, or lymphoma.

45. The method of claim 44, wherein the leukemia is Acute Myeloid Leukemia, or wherein the lymphoma is Hodgkin's lymphoma or non-Hodgkin's lymphoma.

46.-48. (canceled)

49. The method of claim 43, wherein the solid tumor is a carcinoma, lung cancer, breast cancer, liver cancer, pancreatic cancer, gastric cancer, ovarian cancer, or colon cancer.

50.-51. (canceled)

52. The method of claim 49, wherein the lung cancer is non-small cell lung cancer.

53.-60. (canceled)

61. A method of inducing the degradation of a transcription factor in a subject, the method comprising:
administering to the subject a therapeutically effective amount of the compound or a pharmaceutically acceptable salt, co-crystal, tautomer, stereoisomer, solvate, hydrate, polymorph, isotopically enriched derivative, or prodrug thereof of claim 1, 15, or 38.

62. The method of claim 61, wherein the transcription factor is IKZF1 or IKZF3.

63. (canceled)

64. A method of inducing the degradation of a transcription factor in a cell, tissue, or biological sample, the method comprising:
contacting the cell, tissue, or biological sample with the compound or pharmaceutically acceptable salt, co-crystal, tautomer, stereoisomer, solvate, hydrate, polymorph, isotopically enriched derivative, or prodrug thereof of claim 1, 15, or 38.

65.-98. (canceled)


SEQUENCE LISTING (3 sequences)

SEQ ID NO: 1 - protein, 352 aa, Homo sapiens [sequence omitted]

SEQ ID NO: 2 - protein, 923 aa, Homo sapiens
MEEGNNNEEVIHLNNFHCHRGQEWINLRDGPITISDSSDEERIPMLVTPAPQQHEEEDLDDDVILTETNKPQRSRPNLIKPAAQWQDLKRLGEERPKKSRAAFESDKSSYFSVCNNPLFDSGAQDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRF

SEQ ID NO: 3 - protein, 866 aa, Homo sapiens
MEEGNNNEEVIHLNNFHCHRGQEWINLRDGPITISDSSDEERIPMLVTPAPQQHEEEDLDDDVILTEDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRF